(19) United States
(12) Patent Application Publication
(10) Pub. No.: US 2019/0228836 A1
(43) Pub. Date: Jul. 25, 2019

(54) SYSTEMS AND METHODS FOR PREDICTING GENETIC DISEASES

(71) Applicant: SensOmics, Inc., Burlingame, CA (US)

(72) Inventors: Sai Zhang, Palo Alto, CA (US); Jingjing Li, Stanford, CA (US); Michael P. Snyder, Stanford, CA (US)

(21) Appl. No.: 16/248,314

(22) Filed: Jan. 15, 2019

Related U.S. Application Data
(60) Provisional application No. 62/617,604, filed on Jan. 15, 2018.
(60) Provisional application No. 62/632,842, filed on Feb. 20, 2018.

(51) Int. Cl. G16B 20/20

(52) U.S. Cl. CPC G16B 20/20

(57) ABSTRACT

The disclosure relates to systems, software and methods for classifying exomic markers, including diagnosing or prognosticating genetic disorders, e.g., autism spectrum disorder or cancer, in a subject based on the detection of the markers in the subject's sample.

### CROSS-REFERENCE TO RELATED APPLICATIONS

This application claims the benefit of U.S. Provisional Application No. 62/617,604, filed on Jan. 15, 2018 and U.S. Provisional Application No. 62/632,842, filed on Feb. 20, 2018, the disclosures in which are incorporated herein by reference in their entirety.

### TECHNICAL FIELD

Embodiments of the disclosure generally relate to the field of medical diagnostics. In particular, embodiments of the disclosure relate to compositions, methods, and systems for tumor detection and diagnosis.

### BACKGROUND

Genetic variations giving rise to single-nucleotide polymorphisms (SNP) or copy number variations (CNV) are the hallmarks of genetic diversity and also serve as anchors for association studies in the field of genomic medicine. Analysis of SNPs occurring within the coding region of a gene, in particular, nonsynonymous base substitutions altering the length and/or the amino acid composition of the encoded polypeptide product, are especially useful, partly due to the fact that such structural changes in the coding regions often translate into phenotypic changes, e.g., differences in physical traits as well as risk of developing various disorders such as cancer, neuropsychiatric disorders (such as schizophrenia), metabolic disorders (such as diabetes) and other genetic defects (such as autism).

One of the key issues regarding limited applicability of genomic studies in the field of complex genetic diseases such as autism rests on the fact that there is little clinical information to guide genetic interpretation. In this context, clinical information regarding association between genes and diseases is generally limited to loss-of-function (LoF) mutations, as these are easier to characterize and also study. In contrast, the effects of missense mutations are largely ignored, perhaps, because the effect of non-LoF missense mutations on the phenotype is often not singular or binary. That is, unlike LoF mutations, the effect of non-LoF missense mutations is often cumulative. Also, since many missense mutations are only mildly deleterious and effectuate very subtle phenotypic changes, if any, they are difficult to characterize clinically. Various studies analyzing the genetic basis of complex human diseases have observed high incidence rates of missense mutations in the genomes of the afflicted subjects. In fact, the accumulation of mildly deleterious missense mutations in individual human genomes has been proposed to be a genetic basis for complex diseases. See, Kryukov et al., Am J Hum Genetics, 80(4):727-39, 2007. However, existing systems for analysis of these missense mutations are lacking. For example, a polymorphism phenotyping tool (POLYPHEN version 2) developed by Adzhubei et al. (see, Curr Protoc Hum Genet., Chapter 7:Unit7.20, 2013 and Nat Methods 7(4):248-249, 2010) is highly aggressive, predicting nearly half of the missense mutations being deleterious, which is unlikely in practice.

Recent advances in computer-based machine learning technologies have enabled scientists to accommodate large quantities of data derived from complex in vivo systems and apply them to analyze features associated with genetic disorders. In general, machine learning algorithms are often configured to identify patterns in training data sets so that the algorithms “learn” or become “trained” how to predict possible outcomes when presented with new input data. Notably, there are numerous types of machine learning algorithms, each having their own specific underlying mode of analysis (e.g., support vector machines, Bayesian statistics, Random Forests, etc.), and with an inherent bias. See, e.g., U.S. Pat. Nos. 7,321,881; 7,467,119; 7,505,948; 7,617,163; 7,676,442; 7,702,598; 7,707,134; and 7,747,547. Specific models and statistical methods based thereon have been devised for prediction or prognosis in a healthcare setting. See, Cesano et al. (US pub. No. 2014/0199273). However, these art-existing models and systems are not tailored to analyze exomic markers and are biased towards analysis of genomic reads. The existing systems do not integrate proteomic features into the screening algorithms such that specific mutation signatures that have a high probability of being associated with a disorder can be identified in a compendium of missense or loss-of-function markers.

There is therefore an unmet need for an automated mutation annotation system that is accurate, probabilistic and also fast compared to the existing systems and methods.

### SUMMARY

The disclosure meets the foregoing needs and provides methods, systems, and devices to quickly and accurately analyze genetic data and identify novel or undiscovered markers therein, which results in superior data research, such as identification of markers that are associated with complex phenotypic traits such as human diseases. The methods and systems of the disclosure can be applied rigorously to identify at-risk subjects.

In some embodiments, the disclosure relates to use of neural networks to identify phenotypic markers that are associated with the phenotypic traits. In particular, the present disclosure relates to use of an exome profiler (termed “Engine”) for diagnosing and prognosticating complex diseases such as cancer, neuropsychiatric disorders (such as schizophrenia), metabolic disorders (such as diabetes) and other genetic defects (such as autism). Further, once the markers are identified, the specific relationships between the markers and the traits (e.g., disease risk) are applied in clinical diagnostics as well as therapeutic optimization to diagnose, treat and maintain such subjects.

In particular, Engine performed significantly better than art-known mutation classifiers such as Polyphen, M-CAP and CADD with respect to accurately identifying markers that are associated with complex human diseases. For example, in analyzing exome sequences for 2500 autism families (1631 in probands versus 1111 siblings), the Engine of the disclosure outperformed or was at least comparable to many classifiers with regard to identifying deleterious mutations in the exome data. More importantly, the deleterious mutations which were identified by the system and methods of the disclosure were deemed to impose increased mutation burden in ASD probands, which demonstrates that the markers identified in accordance with disclosure have greater diagnostic and prognostic significance compared to markers identified using art-known mutation callers. Engine performed particularly well in diagnosing disorders that are strongly associated with single gene mutations, e.g., Timothy's syndrome (associated with CACNA1C), Rett's syndrome (associated with MECP2), tuberous sclerosis (associated with TSC2 and/or TSC1), X-linked mental retardation (XLMR) syndrome (associated with ATRX) and autism (associated with SHANK3). Moreover, analysis of exomes of tumors containing BRCA1/2 or p53 mutations demonstrate that Engine can be readily used in cancer mutational screening and tumor diagnostics.

Furthermore, cross-analysis of existing clinical datasets with Engine validated the significance of genetic markers that previously hypothesized to be associated with various genetic disorders. For example, Engine successfully validated the association between PTEN mutation and macrocephaly, as hypothesized in previous studies. Similarly, Engine was able to validate the association between RAI1 mutations and development of Smith-Magenis Syndrome (SMS). Accordingly, the systems and methods of the disclosure can be used to validate markers identified from genetic association studies and in the case of multifactorial genetic disorders, can also be used to identify high-ranking gene candidates.

The disclosure relates to the following non-limiting embodiments:

In some embodiments, the disclosure relates to a system for diagnosing a genetic disorder, comprising, (a) a receiving unit for receiving a compendium of markers received from a subject's sample, wherein the markers comprise missense mutations in a read; (b) a processing unit comprising one or more processors, each of which is configured to execute computer-readable instructions, which when executed, cause the processor to carry out a method or a set of steps comprising (1) analyzing the compendium of missense mutations for one or more features comprising (I) features relating to protein sequence annotation; (II) features relating to sequence alignment scores; (III) three-dimensional structural features of the encoded protein; (IV) nucleotide sequence context features; or (V) a combination thereof; (2) assigning a classification score to each missense mutation marker based on the number and/or types of missense features associated therewith; (3) assigning a variant score (Sv) to each missense mutation based on the classification score; (4) mapping each missense mutation to one or more genes; and (5) computing a gene score (Sg) based on the peak, mean or median Sv score of the missense mutation mapped thereto and optionally tabulating the genes in the order of decreasing Sg scores; and (c) a diagnosing unit which diagnoses the genetic disorder if the optionally tabulated genes with the highest Sg score(s) are associated with the disorder.

In some embodiments, the system of the foregoing may further comprise (a) a receiving unit for receiving a compendium of markers received from a subject's sample, wherein the markers further comprise Loss-of-Function (LoF) mutations in a read; (b) a processing unit comprising one or more processors configured to execute computer-readable instructions, which when executed, cause the processor to carry out a method or a set of steps further comprising (6) analyzing the compendium of markers comprising LoF mutations for one or more features comprising (VI) probability of intolerant loss of function (pLI) and optionally (VII) proximal positioning of the intolerant LoF mutant marker in the exome sequence; (7) assigning a variant score (Sv) to each LoF mutant marker based on the pLI score and optionally the proximal position score (PS); (8) mapping each LoF mutant marker to one or more genes; and (9) computing a gene score (Sg) based on the peak, mean or median Sv score of the LoF mutant mapped thereto and optionally tabulating the genes in the order of decreasing Sg scores; and (c) a diagnosing unit which diagnoses the genetic disorder if the optionally tabulated genes with the highest Sg score(s) are associated with the disorder.

In some embodiments, in the foregoing system(s) the processor is configured to execute computer-readable instructions, which when executed, cause the processor to carry out a method or a set of steps comprising (1) analyzing the compendium of missense mutations for one or more features comprising (I) protein sequence annotation feature selected from a categorical feature or an integer feature, wherein the categorical features are selected from (1) UNIPROTKB-database derived substitution SITE annotation; (2) UNIPROTKB-database derived substitution REGION annotation; and (3) Pfam identifier of the query protein; and the integer feature comprises (4) UNIPROTKB or Swiss-PROT-database derived PHAT matrix element for substitutions in the transmembrane region; (II) sequence alignment score feature which is a real or categorical, wherein the real feature comprises (1) difference of PSIC scores between two amino acid residue variants; (2) PSIC score for wild type amino acid residue; (3) maximum congruency of the mutant amino acid residue to all sequences in multiple alignment; (4) maximum congruency of the mutant amino acid residue to the sequences in multiple alignment with the mutant residue; (5) query sequence identity with the closest homologue deviating from the wild type amino acid residue; or an integer feature which is (6) number of residues at the substitution position in multiple alignment; (III) three-dimensional structural features of the encoded protein, which are real features, categorical features, or integer features, wherein the real features are selected from (1) sequence identity between query sequence and aligned PDB sequence; (2) normalized accessible surface area; (3) change in solvent accessible surface propensity; (4) normalized B-factor (temperature factor) for the residue; (5) closest residue contact with a heteroatom, Å; (6) closest residue contact with other chain; Å; and (7) closest residue contact with a critical site, Å; and wherein the category features are selected from (8) DSSP secondary structure assignment; and (9) region of the Ramachandran map derived from the residue dihedral angles; and wherein the integer feature selected from (10) change in residue side chain volume; (11) number of hydrogen sidechain-sidechain and sidechain-mainchain bonds formed by the residue; (12) number of residues in contacts with heteroatoms, average per homologous PDB chain; (13) number of residue contacts with other chains, average per homologous PDB chain; and (14) number of residue contacts with critical sites, average per homologous PDB chain; and/or (IV) nucleotide sequence context features, which are binary features, categorical features, or integer features, wherein, the binary features comprise (1) assessment of transversions; wherein categorical features comprise (2) assessment of position of the substitution within a codon; or (3) substitution changes CpG context; and wherein the integer feature comprises (4) assessment of the substitution distance from closest exon/intron junction.

In some embodiments, in the foregoing system(s), the diagnosing unit comprises a neural network which is capable of identifying markers associated with the disorder from a training dataset generated from a genetic data of a patient diagnosed with the disorder or a subject related thereto, wherein the training dataset comprises a compendium of markers that are prognostic of the disease.

In some embodiments, the disclosure relates to methods for diagnosing a genetic disorder, comprising, (a) receiving in a compendium of markers received from a subject's sample, wherein the markers comprise missense mutations in an read; (b) implementing a plurality of computer-assisted analytical steps comprising (1) analyzing the compendium of missense mutations for one or more features comprising (I) features relating to protein sequence annotation; (II) features relating to sequence alignment scores; (III) three-dimensional structural features of the encoded protein; (IV) nucleotide sequence context features; or (V) a combination thereof; (2) assigning a classification score to each missense mutation marker based on the number and/or types of missense features associated therewith; (3) assigning a variant score (Sv) to each missense mutation based on the classification score; (4) mapping each missense mutation to one or more genes; and (5) computing a gene score (Sg) based on the peak, mean or median Sv score of the missense mutation mapped thereto and optionally tabulating the genes in the order of decreasing Sg scores; and (c) diagnosing the genetic disorder if the optionally tabulated genes with the highest Sg score(s) are associated with the disorder.

In some embodiments, the disclosure relates to the foregoing diagnostic methods, wherein step (a) further comprises receiving compendium of markers received from a subject's sample, wherein the markers further comprise Loss-of-Function (LoF) mutations in an read; step (b) further implementing a plurality of computer-assisted analytical steps comprising (6) analyzing the compendium of markers comprising LoF mutations for one or more features comprising (VI) probability of intolerant loss of function (pLI) and optionally (VII) proximal positioning of the intolerant LoF mutant marker in the exome sequence; (7) assigning a variant score (Sv) to each LoF mutant marker based on the pLI score and optionally the proximal position score (PS); (8) mapping each LoF mutant marker to one or more genes; and (9) computing a gene score (Sg) based on the peak, mean or median Sv score of the LoF mutant mapped thereto and optionally tabulating the genes in the order of decreasing Sg scores; and step (c) further comprises diagnosing the genetic disorder if the optionally tabulated genes with the highest Sg score(s) are associated with the disorder.

In some embodiments, the disclosure relates to the foregoing diagnostic methods, wherein the computer-assisted method comprises analyzing the compendium of missense mutations for one or more features comprising (I) protein sequence annotation feature selected from a categorical feature or an integer feature, wherein the categorical features are selected from (1) UNIPROTKB-database derived substitution SITE annotation; (2) UNIPROTKB-database derived substitution REGION annotation; and (3) Pfam identifier of the query protein; and the integer feature comprises (4) UNIPROTKB or Swiss-PROT-database derived PHAT matrix element for substitutions in the transmembrane region; (II) sequence alignment score feature which is a real or categorical, wherein the real feature comprises (1) difference of PSIC scores between two amino acid residue variants; (2) PSIC score for wild type amino acid residue; (3) maximum congruency of the mutant amino acid residue to all sequences in multiple alignment; (4) maximum congruency of the mutant amino acid residue to the sequences in multiple alignment with the mutant residue; (5) query sequence identity with the closest homologue deviating from the wild type amino acid residue; or an integer feature which is (6) number of residues at the substitution position in multiple alignment; (III) three-dimensional structural features of the encoded protein, which are real features, categorical features, or integer features, wherein the real features are selected from (1) sequence identity between query sequence and aligned PDB sequence; (2) normalized accessible surface area; (3) change in solvent accessible surface propensity; (4) normalized B-factor (temperature factor) for the residue; (5) closest residue contact with a heteroatom, AÅ; (6) closest residue contact with other chain; Å; and (7) closest residue contact with a critical site, Å; and wherein the category features are selected from (8) DSSP secondary structure assignment; and (9) region of the Ramachandran map derived from the residue dihedral angles; and wherein the integer feature selected from (10) change in residue side chain volume; (11) number of hydrogen sidechain-sidechain and sidechain-mainchain bonds formed by the residue; (12) number of residues in contacts with heteroatoms, average per homologous PDB chain; (13) number of residue contacts with other chains, average per homologous PDB chain; and (14) number of residue contacts with critical sites, average per homologous PDB chain; and/or (IV) nucleotide sequence context features, which are binary features, categorical features, or integer features, wherein, the binary features comprise (1) assessment of transversions; wherein categorical features comprise (2) assessment of position of the substitution within a codon; or (3) substitution changes CpG context; and wherein the integer feature comprises (4) assessment of the substitution distance from closest exon/intron junction.

In some embodiments, the disclosure relates to the foregoing diagnostic methods, wherein the diagnosing step (c) comprises implementing a neural network to analyze the markers, wherein the neural network is trained with a dataset generated from a genetic data of a patient diagnosed with the disorder or a subject related thereto.

In some embodiments, the disclosure relates to computer readable medium comprising computer-executable instructions, which, when executed by a processor, cause the processor to carry out a method or a set of steps for analyzing a plurality of variant exomic markers contained in a dataset, wherein the dataset is obtained by sequencing a biological sample comprising nucleic acid molecules from a subject afflicted with a disorder, the steps for analyzing the variant exomic markers comprise (a) receiving in a compendium of markers received from a subject's sample, wherein the markers comprise missense mutations in an read; (b) implementing a plurality of computer-assisted analytical steps comprising (1) analyzing the compendium of missense mutations for one or more features comprising (I) features relating to protein sequence annotation; (II) features relating to sequence alignment scores; (III) three-dimensional structural features of the encoded protein; (IV) nucleotide sequence context features; or (V) a combination thereof; (2) assigning a classification score to each missense mutation marker based on the number and/or types of missense features associated therewith; (3) assigning a variant score (Sv) to each missense mutation based on the classification score; (4) mapping each missense mutation to one or more genes; and (5) computing a gene score (Sg) based on the peak, mean or median Sv score of the missense mutation mapped thereto and optionally tabulating the genes in the order of decreasing Sg scores; and (c) diagnosing the genetic disorder if the optionally tabulated genes with the highest Sg score(s) are associated with the disorder.

In some embodiments, the disclosure relates to computer readable medium comprising computer-executable instructions, which, when executed by a processor, cause the processor to carry out a method or a set of steps for analyzing a plurality of variant exomic markers contained in a dataset, wherein the analytical method comprises (a) receiving compendium of markers received from a subject's sample, wherein the markers comprise missense mutations in an read and Loss-of-Function (LoF) mutations in an read; (b) implementing a plurality of computer-assisted analytical steps comprising (1) analyzing the compendium of missense mutations for one or more features comprising (I) features relating to protein sequence annotation; (II) features relating to sequence alignment scores; (III) three-dimensional structural features of the encoded protein; (IV) nucleotide sequence context features; or (V) a combination thereof; (2) assigning a classification score to each missense mutation marker based on the number and/or types of missense features associated therewith; (3) assigning a variant score (Sv) to each missense mutation based on the classification score; (4) mapping each missense mutation to one or more genes; and (5) computing a gene score (Sg) based on the peak, mean or median Sv score of the missense mutation mapped thereto and optionally tabulating the genes in the order of decreasing Sg scores; and further (6) analyzing the compendium of markers comprising LoF mutations for one or more features comprising (VI) probability of intolerant loss of function (pLI) and optionally (VII) proximal positioning of the intolerant LoF mutant marker in the exome sequence; (7) assigning a variant score (Sv) to each LoF mutant marker based on the pLI score and optionally the proximal position score (PS); (8) mapping each LoF mutant marker to one or more genes; and (9) computing a gene score (Sg) based on the peak, mean or median Sv score of the LoF mutant mapped thereto and optionally tabulating the genes in the order of decreasing Sg scores; and (c) diagnosing the genetic disorder if the optionally tabulated genes with the highest Sg score(s) are associated with the disorder.

In some embodiments, the disclosure relates to computer readable medium comprising computer-executable instructions, which, when executed by a processor, cause the processor to carry out a method or a set of steps for analyzing a plurality of variant exomic markers contained in a dataset, wherein the dataset is obtained by sequencing a biological sample comprising nucleic acid molecules from a subject afflicted with a disorder, the steps for analyzing the variant exomic markers comprise (a) receiving in a compendium of markers received from a subject's sample, wherein the markers comprise missense mutations in an read; (b) implementing a plurality of computer-assisted analytical steps comprising (1) analyzing the compendium of missense mutations for one or more features comprising (I) protein sequence annotation feature selected from a categorical feature or an integer feature, wherein the categorical features are selected from (1) UNIPROTKB-database derived substitution SITE annotation; (2)

UNIPROTKB-database derived substitution REGION annotation; and (3) Pfam identifier of the query protein; and the integer feature comprises (4) UNIPROTKB or Swiss-PROT-database derived PHAT matrix element for substitutions in the transmembrane region; (II) sequence alignment score feature which is a real or categorical, wherein the real feature comprises (1) difference of PSIC scores between two amino acid residue variants; (2) PSIC score for wild type amino acid residue; (3) maximum congruency of the mutant amino acid residue to all sequences in multiple alignment; (4) maximum congruency of the mutant amino acid residue to the sequences in multiple alignment with the mutant residue; (5) query sequence identity with the closest homologue deviating from the wild type amino acid residue; or an integer feature which is (6) number of residues at the substitution position in multiple alignment; (III) three-dimensional structural features of the encoded protein, which are real features, categorical features, or integer features, wherein the real features are selected from (1) sequence identity between query sequence and aligned PDB sequence; (2) normalized accessible surface area; (3) change in solvent accessible surface propensity; (4) normalized B-factor (temperature factor) for the residue; (5) closest residue contact with a heteroatom, Å; (6) closest residue contact with other chain; Å; and (7) closest residue contact with a critical site, Å; and wherein the category features are selected from (8) DSSP secondary structure assignment; and (9) region of the Ramachandran map derived from the residue dihedral angles; and wherein the integer feature selected from (10) change in residue side chain volume; (11) number of hydrogen sidechain-sidechain and sidechain-mainchain bonds formed by the residue; (12) number of residues in contacts with heteroatoms, average per homologous PDB chain; (13) number of residue contacts with other chains, average per homologous PDB chain; and (14) number of residue contacts with critical sites, average per homologous PDB chain; and/or (IV) nucleotide sequence context features, which are binary features, categorical features, or integer features, wherein, the binary features comprise (1) assessment of transversions; wherein categorical features comprise (2) assessment of position of the substitution within a codon; or (3) substitution changes CpG context; and wherein the integer feature comprises (4) assessment of the substitution distance from closest exon/intron junction.

In some embodiments, the disclosure relates to classifier for classifying a plurality of variant exomic markers contained in a dataset which are received from a subject's sample, wherein the markers comprise missense mutations in an read, the neural network capable of implementing a plurality of computer-assisted analytical steps comprising (1) analyzing the compendium of missense mutations for one or more features comprising (I) features relating to protein sequence annotation; (II) features relating to sequence alignment scores; (III) three-dimensional structural features of the encoded protein; (IV) nucleotide sequence context features; or (V) a combination thereof. In some embodiments, the classifier comprises support vector machines (SVMs), logistic regression, random forest, naïve Bayes, gradient boosting, or neural network (NN), preferably neural networks.

In some embodiments, the disclosure relates to the foregoing classifiers, which implement a three-layer feed-forward neural network which models the features of Table 2 in n dimensions.

In some embodiments, the disclosure relates to the foregoing classifiers, which implement a three-layer feed-forward neural network which models the features of Table 2 in n dimensions, wherein the neural network further comprises two hidden layers.

The disclosure further relates to use of the foregoing classifiers, computer programs and/or systems for diagnosing, prognosticating, and/or nutritional or therapeutic intervention of genetic disorders, for example, human genetic disorders such as, e.g., Timothy's syndrome; Rett's syndrome; tuberous sclerosis; cancer; X-linked mental retardation syndrome; autism; Smith-Magenis syndrome; macrocephaly, or a combination thereof.

### BRIEF DESCRIPTION OF THE DRAWINGS

The details of one or more embodiments of the disclosure are set forth in the accompanying drawings/tables and the description below. Other features, objects, and advantages of the disclosure will be apparent from the drawings/tables and detailed description, and from the claims.

FIG. 1 shows a schematic chart of the exome analyzer of the instant disclosure. A compendium of markers contained in whole exome sequence dataset (e.g., VCF file) is received and fed to the analyzer. The analyzer includes a module (PATH) for determining whether the markers are pathogenic or not. Typically, this is accomplished by analyzing the markers in ClinVar database. Markers which are deemed to be pathogenic are then fed into a second frequency analyzer module. The frequency analyzer module determines whether the marker is rare or common (R/C). Typically, the frequency analyzer includes use of EXAC database. In some embodiments, the frequency analyzer may include 1000 Genomes database (available on the web at internationalgenome(dot)org). Only markers deemed to be rare are selected for further analysis, in accordance with the flow chart of FIG. 2. The output of the analysis is score table listing the individual markers and their scores.

FIG. 2 shows a flow chart used in analysis of the rare, pathogenic markers that are obtained by the Exome Analyzer of FIG. 1.

FIG. 3 provides details on the computational processes used in assigning weights to the rare, pathogenic markers.

FIG. 4 shows a diagram of the computer system of the present disclosure.

FIG. 5 shows receiving operator characteristic (ROC) curves of the markers analyzed by the systems/methods of the disclosure. Genetic markers contained in a benchmarked dataset were annotated as true positives versus false positives and analyzed. The outputs of two other analytical tools—POLYPHEN version 2 and POLYPHEN variant—are included for comparative purposes. The results show that the methods/systems of the disclosure perform better than POLYPHEN, as evidenced by the greater area under the ROC curve (AUC). More specifically, the Engine (DNN) of the disclosure permits identification of true positive markers at the highest rate without concomitantly including false positives in the dataset.

FIG. 6 and FIG. 7, each independently, show empirical cumulative distribution function (CDF) of various analytical tools. Each curve shown in the figures represents CDFs, wherein Fx is the probability of the prediction smaller than (x). FIG. 6 shows comparative CDFs of predictions made by Engine in comparison to two versions of Polyphen, Polyphen VAR and Polyphen DIV. FIG. 7 shows comparative assessments between Engine and the aforementioned Polyphen tools, and also M-CAP and CADD.

FIG. 8 shows a bar chart of mutation burden analysis, as identified by Engine versus Polyphen.

FIG. 9 shows a bar chart of mutation burden analysis, as identified by Engine vs. Polyphen2 (two types used in the assessment, HVAR and HDIV) vs. M-CAP vs. CADD (two levels used in the assessment, at 0.5 and 0.95).

FIG. 10A-10D show ROC curves for the recited genetic markers and their association with various diseases, as analyzed using the Engine of the present disclosure. FIG. 10A shows ROC curve for the association between CACNA1C and Timothy's syndrome. FIG. 10B shows ROC curve for the association between MECP2 and Rett' s syndrome. FIG. 10C shows ROC curve for the association between TSC2 and tuberous sclerosis. FIG. 10D shows ROC curve for the association between various DNA damage/checkpoint proteins and cancer, e.g., BRCA1 and cancer (top); BRCA2 and cancer (middle) and p53 and cancer (bottom).

FIG. 11 shows mutational screening result for 19 autism patients, and highlighted one patient with one disrupted gene phosphatase and tensin homolog (PTEN). The PTEN gene disruption comprises a gain a stop codon, resulting in a premature transcript.

FIG. 12 shows association between mutations in phosphatase and tensin homolog (PTEN) protein and macrocephaly, as analyzed using the Engine of the present disclosure.

FIG. 13 shows association between mutations retinoic acid induced 1 (RAI1) and Smith-Magenis Syndrome.

FIG. 14 shows a representative study protocol used in the evaluation of the linkage between genetic markers and autism spectrum disorder (ASD) in accordance with the methods of the present disclosure. Briefly, ten individuals recruited during their clinic visits. The age of the subjects are between 3 years-4.4 years. The primary criteria for inclusion is M-CHAT-R positive (cutoff-score is 3). The subjects are undiagnosed, i.e., no confirmatory ASD diagnosis. Also, the patient cohort is unselected.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are purely representative and do not limit the disclosure.

FIG. 15 shows details of the workflow/pipeline using Profiler and Engine (DEEPSCAN). In the pipeline ‘B’, T′, ‘C’, ‘R’ and ‘LoF’ represent benign, pathogenic, common, rare and loss of function, respectively. The pLI score indicates the probability that a gene is intolerant to a loss of function mutation, which in turn, correlates with functional effects of missense variants of the gene.

### DETAILED DESCRIPTION

The present disclosure will now be described in more detail with reference to the accompanying drawings, in which preferred embodiments of the disclosure are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

Unless otherwise defined, scientific and technical terms used in connection with the present teachings described herein shall have the meanings that are commonly understood by those of ordinary skill in the art. The terminology used in the description of the disclosure herein is for the purpose of describing particular embodiments only and is not intended to be limiting of the disclosure. Further, unless otherwise required by context, singular terms shall include pluralities and plural terms shall include the singular. Generally, nomenclatures utilized in connection with, and techniques of molecular biology, and protein and oligo- or polynucleotide chemistry and hybridization described herein are those well-known and commonly-used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly-used in the art.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be expressly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following descriptions.

The methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software, including, software on cloud. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Whole exome sequencing technology enables research on a large scale. Particularly, the methods and systems of the disclosure can utilize de-identified, clinical information and biological data for medically relevant associations. The methods and systems disclosed can comprise a high-throughput platform for discovering and validating genetic factors that cause or influence a range of diseases, including diseases where there are major unmet medical needs.

The various embodiments of the present disclosure are further described in detail in the paragraphs below.

### I. Definitions

As used in the description of the disclosure and the appended claims, the singular forms “a,” “an,” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The word “about” means a range of plus or minus 10% of that value, e.g., “about 5” means 4.5 to 5.5, “about 100” means 90 to 100, etc., unless the context of the disclosure indicates otherwise, or is inconsistent with such an interpretation. For example in a list of numerical values such as “about 49, about 50, about 55”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

Where a range of values is provided in this disclosure, it is intended that each intervening value between the upper and lower limit of that range and any other stated or intervening value in that stated range is encompassed within the disclosure. For example, if a range of 1 μM to 8 μM is stated, it is intended that 2 μM, 3 μM, 4 μM, 5 μM, 6 μM, and 7 μM are also explicitly disclosed.

As used herein, “biological data” can refer to any data derived from measuring biological conditions of human, animals or other biological organisms including microorganisms, viruses, plants and other living organisms. The measurements may be made by any tests, assays or observations that are known to physicians, scientists, diagnosticians, or the like. Biological data can include, but is not limited to, clinical tests and observations, physical and chemical measurements, genomic determinations, genomic sequencing data, exome sequencing data, proteomic determinations, drug levels, hormonal and immunological tests, neurochemical or neurophysical measurements, mineral and vitamin level determinations, genetic and familial histories, and other determinations that may give insight into the state of the individual or individuals that are undergoing testing. As used herein, “phenotypic data” refer to data about phenotypes. Phenotypes are discussed further below.

As used herein, the term “subject” means an individual. In one aspect, a subject is a mammal such as a human. In one aspect a subject can be a non-human primate. Non-human primates include marmosets, monkeys, chimpanzees, gorillas, orangutans, and gibbons, to name a few. The term “subject” also includes domesticated animals, such as cats, dogs, etc., livestock (e.g., cows, pigs, goats), laboratory animals (e.g., mouse, rabbit, rat, gerbil, guinea pig, etc.) and avian species (e.g., chickens, turkeys, ducks, etc.). Subjects can also include, but are not limited to fish (for example, zebrafish, goldfish, tilapia, salmon, and trout), amphibians and reptiles. Preferably, the subject is a human subject. Especially, the subject is a human patient.

The terms “polynucleotide” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide” and “nucleic acid molecule.”

“Nucleotide” as used herein refers to molecules that, when joined, make up the individual structural units of the nucleic acids RNA and DNA. A nucleotide is composed of a nucleobase (nitrogenous base), a five-carbon sugar (either ribose or 2-deoxyribose), and one phosphate group. “Nucleic acids” as used herein are polymeric macromolecules made from nucleotide monomers. In DNA, the purine bases are adenine (A) and guanine (G), while the pyrimidines are thymine (T) and cytosine (C). RNA uses uracil (U) in place of thymine (T).

As used herein, a “nucleic acid,” “polynucleotide,” or “oligonucleotide” can be a polymeric form of nucleotides of any length, can be DNA or RNA, and can be single- or double-stranded. Nucleic acids can include promoters or other regulatory sequences. Oligonucleotides can be prepared by synthetic means. Nucleic acids include segments of DNA, or their complements spanning or flanking any one of the polymorphic sites. The segments can be between 5 and 100 contiguous bases and can range from a lower limit of 5, 10, 15, 20, or 25 nucleotides to an upper limit of 10, 15, 20, 25, 30, 50, or 100 nucleotides (where the upper limit is greater than the lower limit). Nucleic acids between 5-10, 5-20, 10-20, 12-30, 15-30, 10-50, 20-50, or 20-100 bases are common. A reference to the sequence of one strand of a double-stranded nucleic acid defines the complementary sequence and except where otherwise clear from context, a reference to one strand of a nucleic acid also refers to its complement. Complementation can occur in any manner, e.g., DNA=DNA; DNA=RNA; RNA=DNA; RNA=RNA, wherein in each case, the “=” indicates complementation. Complementation can occur between two strands or a single strand of the same or different molecule.

A nucleic acid may be naturally or non-naturally polymorphic, e.g., having one or more sequence differences (e.g., additions, deletions and/or substitutions) as compared to a reference sequence. A reference sequence may be based on publicly available information (e.g., the U.C. Santa Cruz Human Genome Browser Gateway or the NCBI website or may be determined by a practitioner of the present invention using methods well known in the art (e.g., by sequencing a reference nucleic acid).

The term “polymorphism” as used herein refers to the occurrence of one or more genetically determined alternative sequences or alleles in a population. A “polymorphic site” is the locus at which sequence divergence occurs. Polymorphic sites have at least one allele. A diallelic polymorphism has two alleles. A triallelic polymorphism has three alleles. Diploid organisms may be homozygous or heterozygous for allelic forms. A polymorphic site can be as small as one base pair. Examples of polymorphic sites include: restriction fragment length polymorphisms (RFLPs), variable number of tandem repeats (VNTRs), hypervariable regions, minisatellites, dinucleotide repeats, trinucleotide repeats, tetranucleotide repeats, and simple sequence repeats. As used herein, reference to a “polymorphism” can encompass a set of polymorphisms (i.e., a haplotype). A “single nucleotide polymorphism (SNP)” can occur at a polymorphic site occupied by a single nucleotide, which is the site of variation between allelic sequences. The site can be preceded by and followed by highly conserved sequences of the allele. A SNP can arise due to substitution of one nucleotide for another at the polymorphic site. Replacement of one purine by another purine or one pyrimidine by another pyrimidine is called a transition. Replacement of a purine by a pyrimidine or vice versa is called a transversion. A synonymous SNP refers to a substitution of one nucleotide for another in the coding region that does not change the amino acid sequence of the encoded polypeptide. A non-synonymous SNP refers to a substitution of one nucleotide for another in the coding region that changes the amino acid sequence of the encoded polypeptide. A SNP may also arise from a deletion or an insertion of a nucleotide or nucleotides relative to a reference allele.

A nucleic acid polymorphism is characterized by two or more “alleles”, or versions of the nucleic acid sequence. Typically, an allele of a polymorphism that is identical to a reference sequence is referred to as a “reference allele” and an allele of a polymorphism that is different from a reference sequence is referred to as an “alternate allele,” or sometimes a “variant allele”. As used herein, the term “major allele” refers to the more frequently occurring allele at a given polymorphic site, and “minor allele” refers to the less frequently occurring allele, as present in the general or study population.

As used herein, the term “haplotype” refers to a set of two or more alleles (specific nucleic acid sequences) that are in linkage disequilibrium. In one aspect, a haplotype refers to a set of single nucleotide polymorphisms (SNPs) found to be statistically associated with each other on a single chromosome. A haplotype can also refer to a combination of polymorphisms (e.g., SNPs) and other genetic markers (e.g., an insertion or a deletion) found to be statistically associated with each other on a single chromosome.

As used herein, the term “detecting,” refers to the process of determining a value or set of values associated with a sample by measurement of one or more parameters in a sample, and may further comprise comparing a test sample against reference sample. In accordance with the present disclosure, the detection of tumors includes identification, assaying, measuring and/or quantifying one or more markers.

As used herein, the term “diagnosis” refers to methods by which a determination can be made as to whether a subject is likely to be suffering from a given disease or condition, including but not limited diseases or conditions characterized by genetic variations. The skilled artisan often makes a diagnosis on the basis of one or more diagnostic indicators, e.g., a marker, the presence, absence, amount, or change in amount of which is indicative of the presence, severity, or absence of the disease or condition. Other diagnostic indicators can include patient history; physical symptoms (e.g., enlarged brain mass (macrocephaly), distortions in facial tissue/bone structure, abnormal or deformed appendages; diminished motor skills); neurological symptoms (e.g., diminished cognition) phenotype; genotype; or environmental or heredity factors. A skilled artisan will understand that the term “diagnosis” refers to an increased probability that certain course or outcome will occur; that is, that a course or outcome is more likely to occur in a patient exhibiting a given characteristic, e.g., the presence or level of a diagnostic indicator, when compared to individuals not exhibiting the characteristic. Diagnostic methods of the disclosure can be used independently, or in combination with other diagnosing methods, to determine whether a course or outcome is more likely to occur in a patient exhibiting a given characteristic.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

As used herein, the term “sample” refers to a composition that is obtained or derived from a subject of interest that contains a cellular and/or other molecular entity that is to be characterized and/or identified, for example based on physical, biochemical, chemical and/or physiological characteristics. The source of the tissue sample may be blood or any blood constituents; bodily fluids; solid tissue as from a fresh, frozen and/or preserved organ or tissue sample or biopsy or aspirate; and cells from any time in gestation or development of the subject or plasma. Samples include, but not limited to, primary or cultured cells or cell lines, cell supernatants, cell lysates, platelets, serum, plasma, vitreous fluid, ocular fluid, lymph fluid, synovial fluid, follicular fluid, seminal fluid, amniotic fluid, milk, whole blood, urine, cerebrospinal fluid (CSF), saliva, sputum, tears, perspiration, mucus, tumor lysates, and tissue culture medium, as well as tissue extracts such as homogenized tissue, tumor tissue, and cellular extracts. Samples further include biological samples that have been manipulated in any way after their procurement, such as by treatment with reagents, solubilized, or enriched for certain components, such as proteins or nucleic acids, or embedded in a semi-solid or solid matrix for sectioning purposes, e.g., a thin slice of tissue or cells in a histological sample. Preferably, the sample is obtained from blood or blood components, including, e.g., whole blood, plasma, serum, lymph, and the like.

As used herein, the term “marker” refers to a characteristic that can be objectively measured as an indicator of normal biological processes, pathogenic processes or a pharmacological response to a therapeutic intervention, e.g., treatment with an anti-cancer agent. Representative types of markers include, for example, molecular changes in the structure (e.g., sequence) or number of the marker, comprising, e.g., gene mutations, gene duplications, or a plurality of differences, such as somatic alterations in cfDNA, copy number variations, tandem repeats, or a combination thereof.

As used herein the term “exomic marker” refers to a polynucleotide sequence that is translated into a protein product. As is understood in the art, the exome is the part of the genome formed by exons, the sequences which when transcribed remain within the mature RNA after introns are removed by RNA splicing. It comprises all DNA that is transcribed into mature RNA in cells of any type. In contrast, the transcriptome comprises RNA that has been transcribed only in a specific cell population. The exome of the human genome consists of roughly 180,000 exons constituting about 1% of the total genome, or about 30 megabases of DNA (Ng et al., Nature, 461, 272-276, 2009) Though comprising a very small fraction of the genome, mutations in the exome are thought to harbor 85% of mutations that have a large effect on disease (Choi et al., PNAS USA, 106, 19096-19101, 2009). Exome sequencing has proved to be an efficient strategy to determine the genetic basis of more than two dozen Mendelian or single gene disorders (Bamshad et al., Nat Rev Genet., 12, 745-755, 2011).

The term “genetic marker” can also be used to refer to, e.g., a cDNA and/or an mRNA encoded by a genomic sequence, as well as to that genomic sequence itself. Genetic markers may include two or more alleles or variants. Genetic markers may be direct (e.g., located within the gene or locus of interest (e.g., candidate gene)), indirect (e.g., closely linked with the gene or locus of interest, e.g., due to proximity to but not within the gene or locus of interest). Moreover, genetic markers may also be unrelated to the genes or loci, e.g., SNVs, CNVs, or tandem repeats, which are present in non-coding segments of the genome. Genetic markers include nucleic acid sequences which either do or do not code for a gene product (e.g., a protein). Particularly, the genetic markers include single nucleotide polymorphisms/variations (SNPs/SNVs) or copy number variations (CNVs) or a combination thereof.

As used herein, the term “variation” refers to a change or deviation. In reference to nucleic acid, a variation refers to a difference(s) or a change(s) between DNA nucleotide sequences, including differences in copy number (CNVs). This actual difference in nucleotides between DNA sequences may be an SNP, and/or a change in a DNA sequence, e.g., fusion, deletion, addition, repeats, etc., observed when a sequence is compared to a reference, such as, e.g., germline DNA (gDNA) or a reference human genome HG38 sequence. Preferably, the variation refers to difference between sample sequence and a control DNA sequence, such as when a sample sequence is compared to reference HG38 sequence; when a sample sequence is compared to gDNA. Differences identified in both gDNA and cfDNA are considered “constitutional” and may be ignored.

As used herein, the term “altered” in reference to a gene product, e.g., mRNA (or the DNA equivalent thereof or the complement of the mRNA or the DNA equivalent) or a polypeptide encoded by the mRNA or the DNA equivalent, refers to a difference in the structure (e.g., nucleic acid sequence or amino acid sequence), level, activity, or function of the gene product compared to a control. Preferably, the altered gene product comprises missense mutations or loss-of-function (LoF) mutations.

As used herein, the term “genetic variant” or “variant” refers to a nucleotide sequence in which the sequence differs from the sequence most prevalent in a population, for example by one nucleotide, in the case of the SNPs described herein. For example, some variations or substitutions in a nucleotide sequence alter a codon so that a different amino acid is encoded resulting in a genetic variant polypeptide. The term “genetic variant,” can also refer to a polypeptide in which the sequence differs from the sequence most prevalent in a population at a position that does not change the amino acid sequence of the encoded polypeptide (i.e., a conserved change). Genetic variant polypeptides can be encoded by a risk haplotype, encoded by a protective haplotype, or can be encoded by a neutral haplotype. Genetic variant polypeptides can be associated with risk, associated with protection, or can be neutral.

Non-limiting examples of genetic variants include frameshift, stop gained, start lost, splice acceptor, splice donor, stop lost, inframe indel, missense, splice region, synonymous and copy number variants. Non-limiting types of copy number variants include deletions and duplications.

As used herein, “genetic variant data” refer to data obtained by identifying allelic variants in a subject's nucleic acid, relative to a reference nucleic acid sequence. The term “genetic variant data” also encompasses data that represent the predicted effect of a variant on the biochemical structure/function of the polypeptide encoded by the variant gene.

Preferably, the exomic marker or the genetic marker includes variant nucleic acids, e.g., mutations, SNPs, CNVs, STRs, or a combination thereof compared to a reference sample. Particularly, the variations are in the coding region of the nucleic acids, especially in the exomes. The variant nucleic acids preferably encode for an altered protein product, e.g., a protein product whose amino acid composition or length or both is different from a reference (e.g., wild-type) polypeptide product.

As used herein, the term “missense mutation” refers to a change in the DNA sequence that changes a codon in the MRNA that is normally translated as one amino acid into a codon that is translated as a different amino acid. For example, a mutation in which the ‘C’ in 5′-TCA is changed to ‘T’ (UCA to UUA in the mRNA) is a missense mutation. The serine encoded by the TCA codon would be replaced by leucine, the amino acid encoded by the TTA (UUA) codon, when the protein is synthesized in the cell. Some but not all missense mutations result in a non-functional gene-product. Some missense mutations may also result in a gain of function. A selection method may be used to find those missense mutations that substantially affect the protein function.

As used herein, the term “loss-of-function (LoF) mutation” or “inactivating mutation” refers to mutations which result in partial or complete inactivation of the gene product. The term includes “amorphic mutation” which refers to instances wherein an allele has a complete loss of function (null allele). Phenotypes associated with amorphic mutations are most often recessive. Exceptions are when the organism is haploid, or when the reduced dosage of a normal gene product is not enough for a normal phenotype (termed haploinsufficiency). In contrast “gain-of-function (GoF) mutations” or “activating mutations” refers to mutations which enhance activity of the protein product or which result in a wholly different (and abnormal) activity of the protein. When the new allele is created containing a GoF mutation, a heterozygote containing the newly created allele as well as the original allele will express the new allele; genetically this defines the mutations as dominant phenotypes.

In some embodiments, the missense mutations give rise to dominant negative mutations (DN). The term “dominant negative mutation” or “antimorphic mutation” refers to a mutation which results in an altered gene product that acts antagonistically to the wild-type allele. These mutations usually result in an altered molecular function (often inactive) and are characterized by a dominant or semi-dominant phenotype. In humans, dominant negative mutations have been implicated in cancer (e.g., mutations in genes p53, ATM, CEBPA and PPARγ).

As used herein, the term “germline DNA” or “gDNA” refers to DNA isolated or extracted from a subject's germline cells, e.g., peripheral mononuclear blood cells, including lymphocytes that are in turn obtained from circulating blood.

The term “control,” as used herein, refers to a reference for a test sample, such as control DNA isolated from peripheral mononuclear blood cells and lymphocytes, where these cells are not cancer cells, and the like. A “reference sample,” as used herein, refers to a sample of tissue or cells that may or may not have cancer that are used for comparisons. Thus a “reference” sample thereby provides a basis to which another sample, for example plasma sample containing markers, e.g., exomic markers can be compared. In contrast, a “test sample” refers to a sample compared to a reference sample or control sample. In some embodiments, the reference sample or control may comprise a reference assembly.

The term “reference assembly” refers to a digital nucleic acid sequence database, such as the human genome (HG38) database containing HG38 assembly sequences. The gateway can be accessed through the Human (Homo sapiens) University of California Santa Cruz Genome Browser Gateway via the web at genome(dot)ucsc(dot)edu. Alternately, the reference assembly may refer to the Genome Reference Consortium's Human Genomic Assembly (Build #38; Assembled: June, 2017), which is accessible on the internet via the U.S. NCBI website.

In some embodiments, the reference assembly comprises an “exome assembly” or a “transcriptome assembly.” As the name suggests, these refer to a digital nucleic acid sequence database containing the exome or the transcriptome assembly sequences, respectively. In some embodiments, these databases are assembled using a reference assembly such as HG38 assembly sequences. Alternately, institutional exome assemblies can be utilized. An example is Garvan Institute of Medical Research whole-exome sequence data, which is utilized by Illumina' s SEQMAN NGEN 12.2 to analyze Illumina-based sequence data.

As used herein, the term “sequencing” or “sequence” as a verb refers to a process whereby the nucleotide sequence of DNA, or order of nucleotides, is determined, such as a nucleotide order AGTCC, etc. The term “sequence” as a noun refers to the actual nucleotide sequence obtained from sequencing; for example, DNA having the sequence AGTCC. Wherein the “sequence” is provided and/or received in digital form, e.g., in a disk or remotely via a server, “sequencing” may refer to a collection of DNA that is propagated, manipulated and/or analyzed using the methods and/or systems of the disclosure.

The phrase “sequencing run” refers to any step or portion of a sequencing experiment performed to determine some information relating to at least one biomolecule (e.g., nucleic acid molecule).

As used herein the term “whole exome sequencing” refers to selective sequencing of coding regions of the DNA genome. The targeted exome is usually the portion of the DNA that translate into proteins, however regions of the exome that do not translate into proteins may also be included within the sequence. The robust approach to sequencing the complete coding region (exome) can be clinically relevant in genetic diagnosis due to the current understanding of functional consequences in sequence variation, by identifying the functional variation that is responsible for both Mendelian and common diseases without the high costs associated with a high coverage whole-genome sequencing while maintaining high coverage in sequence depth. See, Ng et al., Nature 461, 272-276, 2009 and Choi et al., PNAS USA 106, 19096-19101, 2009.

As used herein the term “whole transcriptome sequencing” refers to determining the expression of all RNA molecules including messenger RNA (mRNA), ribosomal RNA (rRNA), transfer RNA (tRNA), and non-coding RNA. Whole transcriptome sequencing can be done with a variety of platforms for example, the Genome Analyzer (Illumina, Inc., San Diego, Calif., USA) and the SOLID™ Sequencing System (Life Technologies, Carlsbad, Calif., USA). However, any platform useful for whole transcriptome sequencing may be used.

The term “RNA-Seq” or “transcriptome sequencing” refers to sequencing performed on RNA (or cDNA) instead of DNA, where typically, the primary goal is to measure expression levels, detect fusion transcripts, alternative splicing, and other genomic alterations that can be better assessed from RNA. RNA-Seq includes whole transcriptome sequencing as well as target specific sequencing.

The term “whole genome sequencing” or “WGS” refers to a laboratory process that determines the DNA sequence of each DNA strand in a sample. The resulting sequences may be referred to as “raw sequencing data” or “read.” As used herein, a read is a “mappable” read when the sequence has similarity to a region of a reference chromosomal DNA sequence. The term “mappable” may refer to areas that show similarity to and thus “mapped” to a reference sequence, for example, a segment of cfDNA showing similarity to reference sequence in a database, for example, cfDNA having a high percentage of similarity to human chromosomal region 8q248q24.3 in the human genome (HG38) database, is a “mappable read.”

In addition to “WGS,” the genomic compendiums may be obtained using targeted sequencing. In contrast to WGS, the term “targeted sequencing,” as used herein, refers to a laboratory process that determines the DNA sequence of chosen DNA loci or genes in a sample, for example sequencing a chosen group of cancer-related genes or markers (e.g., a target). In this context, the term “target sequence” herein refers to a selected target polynucleotide, e.g., a sequence present in a cfDNA molecule, whose presence, amount, and/or nucleotide sequence, or changes therein, are desired to be determined. Target sequences are interrogated for the presence or absence of a somatic mutation. The target polynucleotide can be a region of gene associated with a disease, e.g., cancer. In some embodiments, the region is an exon.

As used herein, the term “bin” refers to a group of DNA sequences grouped together, such as in a “genomic bin.” In a particular case, the bin may comprise a group of DNA sequences that are binned based on a “genomic bin window,” which includes grouping DNA sequences using genomic windows.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within 10%, or within 5% or less, e.g., with 2%.

As used herein, the term “substantially purified” refers to molecules that are removed from their natural environment, isolated or separated or extracted, and are at least 60% free, preferably 75% free, more preferably 90% free, and most preferably 99% free from other components with which they are naturally associated.

The terms “polypeptide” and “protein” refer to a polymer of amino acid residues and are not limited to a minimum length. Thus, peptides, oligopeptides, dimers, multimers, and the like, are included within the definition. Both full-length proteins and fragments thereof are covered by the definition. The terms also include post-expression modifications of the polypeptide, e.g., glycosylation, acetylation, phosphorylation, hydroxylation, oxidation, and the like.

Methods and systems disclosed herein support large-scale, automated statistical analysis of genetic variant-phenotype associations, on a rolling basis, as genetic variant and phenotype data for new subjects are added over time. For example, in some embodiments, the statistical association analysis that is performed is a genome-wide association study (GWAS) statistical analysis (van der Sluis et al., PLOS Genetics 2013; 9: e1003235; Visscher et al., Am J Hum Genet 2012; 90: 7). In a GWAS analysis, one determines what genes or genetic variants are associated with a phenotype of interest. In some embodiments, the genetic variant data are obtained from genomic sequencing of the subject's sample containing nucleic acids. In another aspect, the genetic variant data are obtained from exome sequencing (e.g., whole exome) of the of the subject's sample containing nucleic acids.

In another aspect, the statistical association analysis that is performed is a phenome-wide association study (PheWAS) statistical analysis (Denny et al., Nature Biotechnol 2013; 31: 1102). In a PheWAS study, one determines phenotypes that are associated with one or more genes or genetic variants of interest. In PheWAS, associations between one or more specific genetic variants and one or more physiological and/or clinical outcomes and phenotypes can be identified and analyzed. In an aspect, algorithms can be utilized to analyze electronic medical record (EMR) and electronic health record (EHR) data. In another aspect, data collected in observational cohort studies can be analyzed.

As used herein, the terms “electronic medical record” and “electronic health record” are synonymous.

As used herein, a genetic variant is “pleiotropic” if it has an effect on more than one phenotype (Gottesman et al., Plos One 7: e46419, 2012). In one embodiment, a genetic variant is associated with an increase in the magnitude of two or more phenotypes, measured, for example, as an increased odds ratio (OR). In another embodiment, a genetic variant is associated with a decrease in the magnitude of two or more phenotypes, measured, for example as a decreased odds ratio. In another embodiment, a genetic variant is associated with an increase in the magnitude of one or more phenotypes and is also associated with a decrease in the magnitude of one or more phenotypes.

In another embodiment, a variant of interest that has been identified in a family affected with a Mendelian disease or in a founder population can be investigated in a larger population for which genetic variant and phenotype information is contained in the present methods and systems. Using that approach, a statistical analysis can be performed to identify what, if any, phenotypes are associated with the variant in a population that is larger than the family affected with a Mendelian disease or the founder population in which the genetic variant was identified. This approach is referred to herein as “family-to-population” analysis.

In another embodiment, a variant of interest that has previously been associated with a phenotype in clinical trial participants can be investigated in a larger population for which genetic variant and phenotype information is contained in the present methods and systems. Using that approach, a statistical analysis can be performed to identify what, if any, phenotypes are associated with the variant in a population that is larger than the group of clinical trial participants.

The present methods and systems also provide a method of gene-based phenotyping. In that method, if a genetic variant-phenotype association has been identified, and if a subject in the population has the variant of interest in the association, but does not exhibit the phenotype of interest associated with the genetic variant, then the subject can be monitored for the development of the phenotype in the future. Alternatively, the subject can be evaluated for the presence of the (previously undiagnosed) phenotype.

Regardless of what type of statistical analysis is employed using the system disclosed, one can filter genetic variant-phenotype association results by any category of interest. Non-limiting categories of interest by which one can filter results are age, sex, race, ethnicity, weight, medicine, diagnosis, laboratory test, laboratory test result, laboratory test result range, or any other phenotype category or type for which the phenotypic data component is configured.

In one embodiment, the genetic variant and phenotype data are obtained from a population of at least 2, 10, 20, 50, 100, 200, 500, 1000, 1500, 2000, 5,000, 10,000, 20,000, 50,000, 100,000, 150,000, 200,000, 250,000, 300,000, 400,000, 500,000 subjects or more, e.g., 1 million, 1.5 million, 5 million, or 10 million subjects. The genetic data and the phenotype data can be used in a statistical analysis of the association of one or more genes and/or one or more genetic variants with one or more phenotypes.

As the sample size (number of sequenced subjects) increases, the number variants found to be significantly associated with one or more phenotypes can increase. To minimize false positive genetic variant-phenotype statistical associations, one must have adequate power and a stringent significance threshold (Sham et al., Nature Rev, 15: 335, 2014). The sample size required for detecting a variant is influenced by both the frequency of the variant, for example the minor allele frequency (MAF), and the effect size of the variant.

In one embodiment, the MAF of a genetic variant is at least 1%, 2%, 3%, 4%, 5%, 6%, 7%, 8%, 9% or 10%. In another embodiment, the MAF of a genetic variant is less than 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02% or 0.01%.

Statistical power depends on allele frequency and effect size. Analysis of rare variants (MAF<1%) can be challenging, due to data sparsity. Even with a large effect size, statistically significant associations for rare variants may only be detected in very large samples. Power may be increased by combining (aggregating) information across variants in a genetic region into a summary dose variable (gene burden testing). Non-limiting examples of gene burden tests are the sequence kernel association test (SKAT), the cohort allelic sum test (CAST), the weighted sum test (WST), the combined multivariate and collapsing method (CMD), the Wald test, and the CMC-Wald test (Wu et al., Am. J. Hum. Genet. 2011; 89: 82; Lee et al., Am. J. Hum. Genet. 2014; 95: 5).

In one embodiment, a phenotype is observed in at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80% or 90%, or more, e.g., 95%, of the subjects from which phenotype information was obtained in the association analysis. In another embodiment, a phenotype is observed in less than 50%, 45%, 40%, 35%, 30%, 25%, 20%, 15%, 10%0, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002% or 0.001% of the subjects from which phenotype information was obtained in the association analysis.

In order to determine the penetrance of a variant of interest on one or more phenotypes of interest in a statistical association study, a case-control study can be performed (Sham et al., Nature Rev, 15: 335, 2014).

In one embodiment, the present methods and systems contain de-identified subject information, which means that neither the genetic data component (which contains a subject's genetic variant data) nor the phenotypic data component (which contains a subject's phenotype data), contain information (such as name, birth date, address, Social Security number, national identifier number, etc.), by which the subject could be identified.

As used herein, a “phenotype” is a clinical designation or category, for example, a clinical diagnosis, a clinical parameter name, a clinical parameter value, a medicine name, dosage or route of administration, a laboratory test name or a laboratory test value. As used herein, a “binary phenotype” is a phenotype that is fixed, i.e., that is either yes or no, for example, a clinical diagnosis, a clinical parameter name, a medicine name or route of administration, or a laboratory test name. As used herein, a “quantitative phenotype” is a phenotype that has a value within a range, for example, a clinical parameter value (for example, a blood pressure value or a serum glucose value), a medicine dosage, or a laboratory test value.

The phenotypic data component can comprise at least 100, 200, 300, 400, 500, 600, 700, 800, 900, 1000, 1100, 1200, 1300, 1400, 1500, 1600, 1700, 1800, 1900 or 2000 categories of phenotypes, among which are at least 100, 200, 300, 400, 500, 600, 700, 800, 900, 1000, 1100, 1200, 1300, 1400, 1500, 1600, 1700, 1800 categories of binary phenotypes and at least 100, 110, 120, 130, 140, 150, 160, 170, 180, 190, 200, 210, 220, 230, 240, 250, 260, 270, 280, 290, 300, 350, 400, 450 or 500 categories of quantitative phenotypes.

As used herein, the term “tumor” is used to denote neoplastic growth which may be benign (e.g., a tumor which does not form metastases and destroy adjacent normal tissue) or malignant/cancer (e.g., a tumor that invades surrounding tissues, and is usually capable of producing metastases, may recur after attempted removal, and is likely to cause death of the host unless adequately treated). See Steadman's Medical Dictionary, 28^{th} Ed Williams & Wilkins, Baltimore, Md. (2005).

As used herein, the term “set” means one or more, e.g., at least 1, at least 2, at least 3, at least 4, at least 5, at least 6, or more than 6 polymorphisms.

As used herein, the term “plurality” can be 2, 3, 4, 5, 6, 7, 8, 9, 10, or more.

As used herein, the term “and/or” includes both conjunctive and disjunctive terms. For instance, the term parameter A and/or parameter B includes, (1) parameter A; OR (2) parameter B; OR (3) parameter A AND parameter B.

Various embodiments are described in detail in the paragraphs below:

### II. Computer Systems

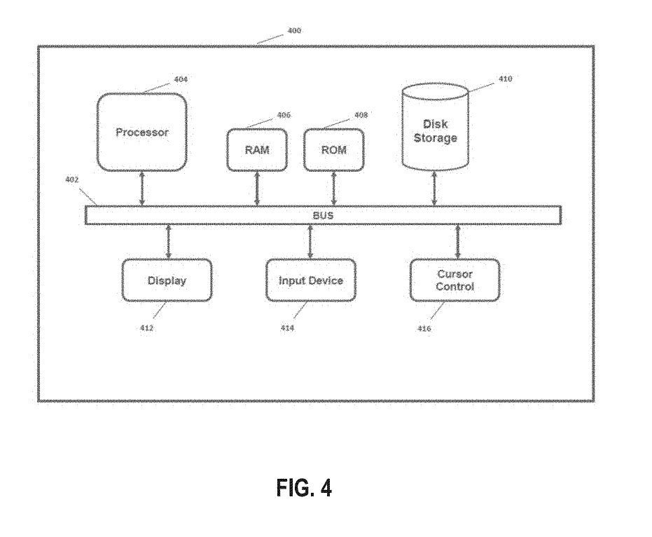

In some embodiments, the diagnostic methods of the disclosure are implemented on a computer system. Purely as a representative example, the schematic representation of such computer systems is provided in FIG. 4. FIG. 4 shows a block diagram that illustrates a computer system 400, upon which, embodiments or portions of the embodiments, of the present disclosure may be implemented. In various embodiments of the present disclosure, computer system 400 can include a bus 402 or other communication mechanism for communicating information, and a processor 404 coupled with bus 402 for processing information. In various embodiments, computer system 400 can also include a memory, which can be a random access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions. In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a trackball or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for 3 dimensional (x, y and z) cursor movement are also contemplated herein.

Consistent with certain implementations of the present disclosure, results can be provided by computer system 400 in response to processor 404 executing one or more sequences of one or more instructions contained in memory 406. Such instructions can be read into memory 406 from another computer-readable medium or computer-readable storage medium, such as storage device 410. Execution of the sequences of instructions contained in memory 406 can cause processor 404 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as memory 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, e.g., telephone modem connections, wide area networks (WAN), local area networks (LAN), infrared data connections, NFC connections, etc.

It should be appreciated that the methodologies described herein, including flow charts, diagrams and accompanying disclosure can be implemented using computer system 400 as a standalone device or on a distributed network of shared computer processing resources such as a cloud computing network.

### III. Methods

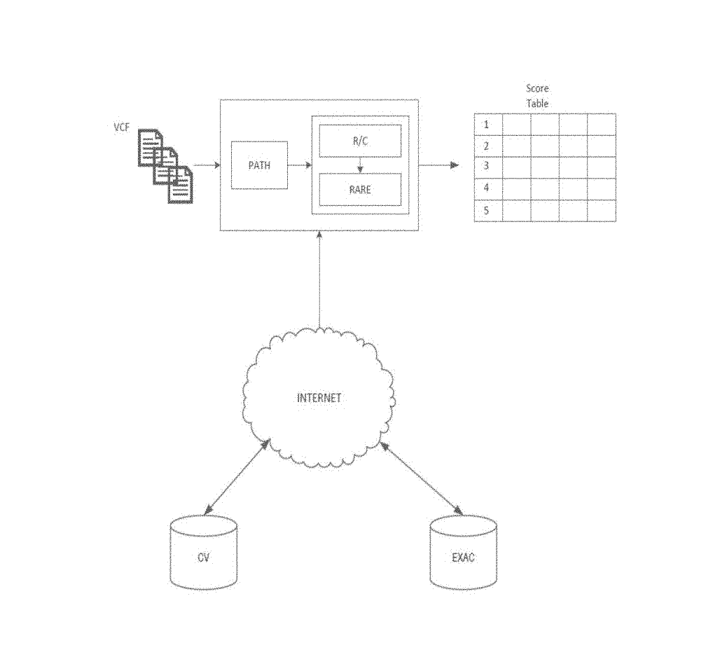

FIG. 1 is a flow chart illustrating a method 100 for diagnosing a genetic disorder (e.g., ASD or cancer) in accordance with the various embodiments of the present disclosure. Method 100 is illustrative only and embodiments can use variations of method 100. Method 100 can include steps for receiving a compendium of markers (e.g., exomic markers obtained by whole exome sequencing, mutation calling, and annotation).

In step 110 of method 100 of FIG. 1, genetic data is received from a subject. In some embodiments, the genetic data comprising a compendium of genetic markers, e.g., exomic markers, is received in a variant call format (VCF) file. As is understood in the art, VCF files are used in bioinformatics for storing gene sequence variations. The VCF format has been developed with the advent of large-scale genotyping and DNA sequencing projects, such as the 1000 Genomes Project. Alternately, the compendium may be provided in a general feature format (GFF) containing all of the genetic data. Generally, GFF provides features that are redundant because they are shared across the genomes. In contrast, with VCF, only the variations need to be stored along with a reference genome. In some embodiments, the subject's sample is sequenced, e.g., using whole genome sequencing (WGS), and the sequence file is processed, e.g., using a tool such as, for example, genome VCF (gVCF).

The received genetic data may be optionally analyzed using a genome toolkit, e.g., Broad Institute's Genome Analysis Toolkit (GATK), ver. 3.3 (McKenna et al., Genome Res., 20: 1297-1303, 2010). The mutations are functionally annotated using compatible programs such as, e.g., ANNOVAR (Wang et al., Nucleic Acids Res., 38(16): e164, 2010).

The genetic data, which are optionally mutation called and/or annotated are then inputted into a pathogenic analyzer (PATH), as shown in 110. A representative example of the pathogenic analyzer is CLINVAR database (maintained by the National Center for Biotechnology Information, U.S. National Library of Medicine available on the web at ncbi(dot)nlm(dot)nih(dot)gov/clinvar). CLINVAR (labeled CV in FIG. 1) is an archive for interpretations of clinical significance of variants for reported conditions and includes germline and somatic variants of any size, type or genomic location (Landrum et al., Nucleic Acids Res., 44(D1):D862-8, 2016). A stand-alone XML file for the database is available via the FTP at ftp(dot)ncbi(dot)nlm(dot)nih(dot)gov/clinvar/xml (last modified: Jan. 4, 2018), the directory containing the file entitled “ClinVarFullRelease_2018-01.xml.gz” and the supplemental materials therein are incorporated by reference herein in its entirety. CLINVAR uses standard terms for clinical significance recommended by American College of Medical Genetics and Genomics-Association for Molecular Pathology (ACMG/AMP) when available. These standards include, benign; likely benign; uncertain; likely pathogenic and pathogenic.

If a marker is of pathogenic significance (e.g., denoted as pathogenic or likely pathogenic by the pathogenic analyzer), it is further examined with a frequency analyzer (FREQ). In some embodiments, the frequency analyzer provides a comprehensive representation of very rare variants and allows for more accurate minor allele frequency (MAF) calculations, which is partly conferred by the dramatically large number of cohorts as well as genetic diversity therein. The frequency analyzer analyzes whether a marker is rare (R) or common (C).

A representative example of the frequency analyzer is Exome Aggregation Consortium (ExAC) catalog (maintained by the Broad Institute and available on the web at exac(dot)broadinstitute(dot)org). EXAC (labeled EXAC in FIG. 1) is a catalogue of high-quality exome DNA sequence data for 60,706 individuals of diverse ancestries (Lek et al., Nature 536, 285-291, 2016). EXAC allows for the direct and accurate characterization of the population burden of pathogenic variants associated with rare Mendelian disorders. A stand-alone file for the catolog can also be obtained via FTP at ftp(dot)broadinstitute(dot)org/pub/ExAC_release (release 1; deposited Jun. 21, 2017), the directory containing the file entitled “ExAC.rl.sites.vep.vcf.gz” and the supplemental materials contained therein (modified: Feb. 26, 2017) are incorporated by reference herein in its entirety.

In some embodiments, the markers are annotated by the frequency analyzer (e.g., ExAC) as loss-of-function mutations (e.g., nonsense, frameshift and consensus splice site variants). Depending on the nature of the frequency analyzer, the annotations may be made as probabilistic outcome, e.g., “HC” (high-confidence) LoF mutation. Variants which are not deemed HC loss-of-function mutations (e.g., missense mutations) are analyzed using a different stream in the pipeline. The details of the pipeline are provided below.

It should be noted that the aforementioned pathogenic analyzer and the frequency analyzer may be implemented in any order, e.g., pathogenic analysis followed by frequency analysis (or vice versa). The analytical steps may be separated in time, e.g., to verify the results of a first analytical step, or may be implemented successively (without significant lag). In some embodiments, the pathogenic analysis is carried out simultaneously with frequency analysis. Herein, “simultaneous” means a gap of less than 10 hours, preferably less than 5 hours.

The product of the analytical method is a table 140 containing markers are outputted on a Table based on the scores they receive using the pipeline of the disclosure. The details of the scoring system are provided in the section below.

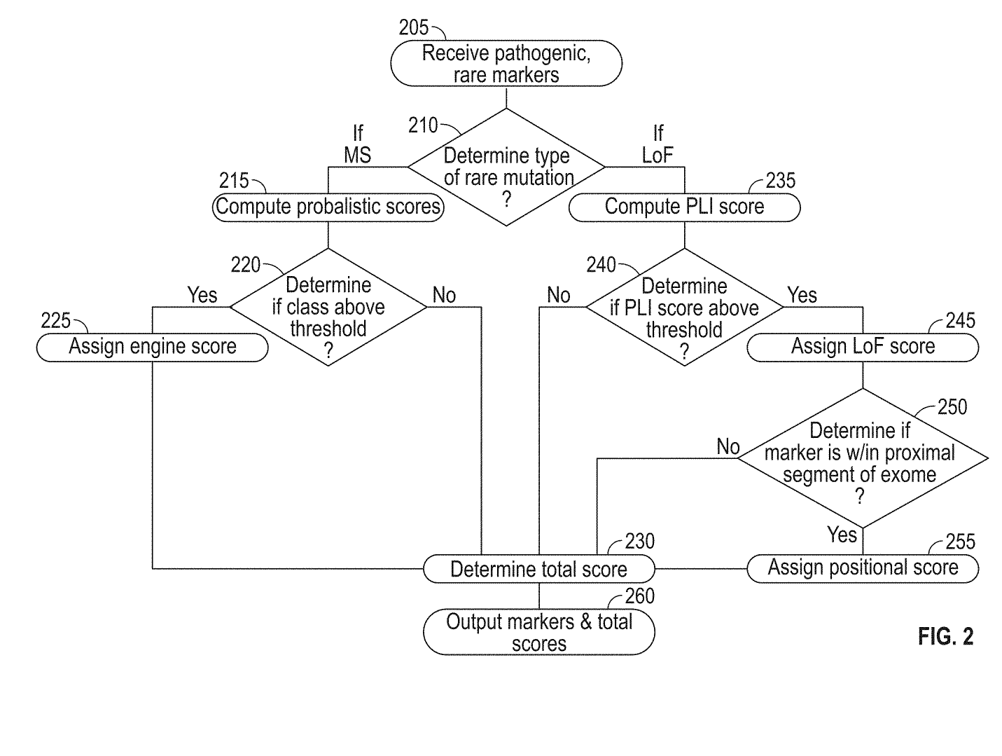

FIG. 2 is a flow chart illustrating a method 200 for the identification of rare, pathogenic markers. As described above, pathogenic exomic markers are identified by a pathogenic analyzer (e.g., using CLINVAR, which characterizes exomic markers as pathogenic or likely pathogenic; collectively termed “pathogenic”). If the markers are not pathogenic, then such markers are eliminated from the dataset. However, if the markers are deemed to be pathogenic, then a variant score (Sv) is assigned for the marker. The Sv score may be any number that is greater than zero. Preferably, the Sv score is between 0.1 and 2.0, e.g., 0.2, 0.4, 0.5, 0.6, 0.8, 1.0, 1.2, 1.4, 1.6, 1.8, 2.0 or more, e.g., 3.0.

Next, markers that are pathogenic are further analyzed using a frequency analyzer (e.g., using EXAC). The frequency analyzer may output its results based on a preset threshold level. For instance, a threshold level may be preset based on the allele frequency, e.g., less than about 10%, 5%, 4%, 3%, 2%, 1%, 0.5%, 0.02%, 0.1%, 0.05%, 0.02%, 0.01%, 0.001% or less, e.g. 0.0001%, or even 0.00001%. If the markers are common (e.g., have an allele frequency greater than the threshold), then the assigned variant score (Sv) for the pathogenic exomic marker is not augmented and such markers are mapped to the gene to determine a gene score (S_{g}), the details of which are described below. However, if the frequency status of an exomic marker on the basis of the EXAC score is rare, then the assigned variant score (Sv) is augmented (e.g., by >0).

Augmentation may be carried out via simple addition or multiplication by a number >1. Preferably, the Sv score for pathogenic, rare markers are augmented by addition of a number between 0.1 and 2.0, e.g., 0.2, 0.4, 0.5, 0.6, 0.8, 1.0, 1.2, 1.4, 1.6, 1.8, 2.0 or more, e.g., 3.0.

Markers are deemed to be pathogenic (e.g., based on CLINVAR annotation of pathogenic or likely pathogenic) and also rare (e.g., have an allele frequency≥the threshold) are entered into a pipeline, which is schematically shown in FIG. 2.

In step 205 of FIG. 2, the type of mutation is determined. In some embodiments, this step is carried out using results obtained from the frequency analyzer. For instance, wherein the analyzer is EXAC, the missense mutations are categorized differently from loss-of-function mutations, which are separately categorized from synonymous mutations and CNVs. As a representative example, EXAC catalog on calcium channel, voltage-dependent, L type, alpha 1C subunit (CACNA1C) is shown below in Table 1:

TABLE 1
EXAC results on CACNA1C

| Constraint | Expected | Observed | Constraint |
|---|---|---|---|
| from ExAC | no. variants | no. variants | Metric |
| Synonymous | 448.1 | 409 | z = 1.14 |
| Missense | 942.9 | 489 | z = 7.23 |
| LoF | 78.3 | 4 | pLI = 1.00 |
| CNV | 9.4 | 0 | z = 1.60 |

As can be seen above, 489 missense mutations have been observed in CACNA1C exome, of which 4 are LoF mutations. To delineate functional effects of the variations, two scoring systems are used—missense mutations are assigned a Z score for the deviation of observed counts from the expected number. Positive Z scores indicate increased constraint (intolerance to variation) and therefore that the gene had fewer variants than expected. Negative Z scores are given to genes that had a more variants than expected. For LoF, EXaC assumes that there are three classes of genes with respect to tolerance to LoF variation: null (where LoF variation is completely tolerated), recessive (where heterozygous LoFs are tolerated), and haploinsufficient (where heterozygous LoFs are not tolerated). EXaC uses the observed and expected variants counts to determine the probability that a given gene is extremely intolerant of loss-of-function variation (falls into the third category). The closer pLI is to one, the more LoF intolerant the gene appears to be. EXaC considers pLI≥0.9 as an extremely LoF intolerant set of genes. Accordingly, it can be seen that CACNA1C is highly intolerant to missense mutation and also highly intolerant to LoF mutations.

Next in step 215, missense markers are classified. Preferably, classification is performed using a computation method comprising support vector machines (SVMs), logistic regression, random forest, naïve Bayes, gradient boosting, or neural network (NN). Preferably, the classification is performed using a convolutional neural network (CNN). The output of such a classifier is a measure of whether the missense mutation is significant.

Next in step 220, missense mutations are analyzed for significance. In some embodiments, significance of a missense mutation is computed on the basis of on one or more features selected from (I) features relating to protein sequence annotation; (II) features relating to sequence alignment scores; (III) three-dimensional structural features of the encoded protein; (IV) nucleotide sequence context features; and/or (V) a combination thereof. For example, the significance of a missense mutation may be computed on the basis of at least 2, at least 3, at least 4, or more of the aforementioned features.

In some embodiments, the features are real or categorical or integer or binary. As is known in statistics, categorical variables comprise classification of data into one or more categories, e.g., blood groups (e.g., A, B, AB or O). Depending on the number of categories and whether there is an ordering to them, the variable is either, binary, nominal, or ordinal. If there are only two categories, then the variable is known as binary (or dichotomous), e.g., yes/no responses. If there are more than two categories and the categories have an obvious order, then the variable is ordinal. Ordinal data may comprise integer (e.g., number of hydrogen sidechain-sidechain and sidechain-mainchain bonds in a polypeptide) or real number (e.g., sequence alignment score in % identity between candidate and reference sequence). Categorical variables which are neither binary nor ordinal are nominal. Nominal measurements do not have meaningful rank order among values, and permit any one-to-one transformation.

The process of encoding categorical data and normalizing numeric data (sometimes called data standardization) can be carried out in accordance with the methods of the present disclosure.

In some embodiments, significance of a missense mutation is computed on the basis of features relating to protein sequence annotation. The feature may be a categorical feature or an integer feature. Representative examples of categorical features include, e.g., (1) UNIPROTKB-database derived substitution SITE annotation (e.g., annotation of wherein the protein sequence the missense mutant product appears); (2) UNIPROTKB-database derived substitution REGION annotation; (3) Pfam identifier of the query protein; and the integer feature comprises (4) UNIPROTKB or Swiss-PROT-database derived PHAT matrix element for substitutions in the transmembrane region.

In some embodiments, significance of a missense mutation is computed on the basis of features relating to sequence alignment scores which is a real or categorical, wherein the real feature comprises (1) difference of PSIC scores between two amino acid residue variants; (2) PSIC score for wild type amino acid residue; (3) maximum congruency of the mutant amino acid residue to all sequences in multiple alignment; (4) maximum congruency of the mutant amino acid residue to the sequences in multiple alignment with the mutant residue; (5) query sequence identity with the closest homologue deviating from the wild type amino acid residue; or an integer feature which is (6) number of residues at the substitution position in multiple alignment.

In some embodiments, significance of a missense mutation is computed on the basis of features relating to three-dimensional structural features of the encoded protein, which are real features, categorical features, or integer features, wherein the real features are selected from (1) sequence identity between query sequence and aligned PDB sequence; (2) normalized accessible surface area; (3) change in solvent accessible surface propensity; (4) normalized B-factor (temperature factor) for the residue; (5) closest residue contact with a heteroatom, Å; (6) closest residue contact with other chain; Å; and (7) closest residue contact with a critical site, Å; wherein the category features are selected from (8) DSSP secondary structure assignment; and (9) region of the Ramachandran map derived from the residue dihedral angles; and wherein the integer feature selected from (10) change in residue side chain volume; (11) number of hydrogen sidechain-sidechain and sidechain-mainchain bonds formed by the residue; (12) number of residues in contacts with heteroatoms, average per homologous PDB chain; (13) number of residue contacts with other chains, average per homologous PDB chain; and (14) number of residue contacts with critical sites, average per homologous PDB chain.

In some embodiments, significance of a missense mutation is computed on the basis of features relating to nucleotide sequence context features, which are binary features, categorical features, or integer features, wherein, the binary features comprise (1) assessment of transversions; wherein categorical features comprise (2) assessment of position of the substitution within a codon; or (3) substitution changes CpG context; and wherein the integer feature comprises (4) assessment of the substitution distance from closest exon/intron junction.

In some embodiments, significance of a missense mutation is computed on the basis of at least 1, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, or all 28 features of Table 2, below.

TABLE 2
Summary of missense features
Protein sequence annotations

| | | |
|---|---|---|
| 1 | category | substitution SITE annotation (UniProtKB/Swiss-Prot derived) |
| 2 | category | substitution REGION annotation (UniProtKB/Swiss-Prot derived) |
| 3 | integer | PHAT matrix element for substitutions in the TRANSMEM region |
| | | (UniProtKB/Swiss-Prot derived) |
| 4 | category | Pfam identifier of the query protein |

Sequence alignment scores

| | | |
|---|---|---|
| 5 | real | difference of PSIC scores between two amino acid residue variants |
| 6 | real | PSIC score for wild type amino acid residue |
| 7 | integer | number of residues at the substitution position in multiple alignment |
| 8 | real | maximum congruency of the mutant amino acid residue to all sequences in |
| | | multiple alignment |
| 9 | real | maximum congruency of the mutant amino acid residue to the sequences in |
| | | multiple alignment with the mutant residue |
| 10 | real | query sequence identity with the closest homologue deviating from the wild type |
| | | amino acid residue |

Protein 3D structural features

| | | |
|---|---|---|
| 11 | real | sequence identity between query sequence and aligned PDB sequence |
| 12 | real | normalized accessible surface area |
| 13 | category | DSSP secondary structure assignment |
| 14 | category | region of the Ramachandran map derived from the residue dihedral angles |
| 15 | integer | change in residue side chain volume |
| 16 | real | change in solvent accessible surface propensity |
| 17 | real | normalized B-factor (temperature factor) for the residue |
| 18 | integer | number of hydrogen sidechain-sidechain and sidechain-mainchain bonds formed |
| | | by the residue |
| 19 | integer | number of residue contacts with heteroatoms, average per homologous PDB chain |
| 20 | real | closest residue contact with a heteroatom, Å |
| 21 | integer | number of residue contacts with other chains, average per homologous PDB chain |
| 22 | real | closest residue contact with other chain, Å |
| 23 | integer | number of residue contacts with critical sites, average per homologous PDB chain |
| 24 | real | closest residue contact with a critical site, Å |

Nucleotide sequence context features

| | | |
|---|---|---|
| 25 | binary | whether substitution is a transversion |
| 26 | category | position of the substitution within a codon |
| 27 | category | whether substitution changes CpG context |
| 28 | integer | substitution distance from closest exon/intron junction |

Next in step 225, the significance of mutations is assessed via an Engine score. Typically, Engine computes significance of the mutations based on one or more of the parameters in Table 2 and outputs a normalized score (e.g., a probability score that the missense mutation is significant). In some embodiments, the Engine score is a normalized score of one or more of the aforementioned parameters, e.g., a score between 0.0 and 1.0, although any range can be used. Markers that have scores that are below a threshold Engine score, e.g., bottom 20%, bottom 30%, bottom 40%, bottom 50%, bottom 60%, bottom 70%, bottom 80%, or even bottom 90%, are not processed further and the total variant score (Sv) of the marker is computed as provided in step 230.

In some embodiments, a series of cutoffs are imposed to bin the Engine scores. In some embodiments, the cutoffs are based on confidence intervals, e.g., about 0% to less than about 50%; about 50% to less than about 80%, about 80% to less than about 95% and greater than about 95%. In some embodiments, a threshold cutoff value of 50% of the normalized Engine score is used as a gate.

Preferably, the threshold Engine score is at least about 0.5 (on a scale of 0.0 to 1.0). Accordingly, markers that have an Engine score below 0.5 are not analyzed further in the pipeline and their Engine scores are computed directly in step 230. Markers that have an Engine score within a predetermined range, e.g., between 0.5 and less than 0.8, are further provided an increase on the Sv score, e.g., of about 0.5. Markers that have an Engine score above this range, e.g., between 0.8 and less than 0.95 are provided a greater increase in the Sv score, of about 1.0. Markers that have an Engine score still further above this range, e.g., greater than 0.95 are provided an even greater increase in the Sv score, of about 2.0.

In some embodiments, each rare, pathogenic, exomic marker is binned as high confidence (HC) missense, medium confidence (MC) missense, low confidence (LC) missense and no confidence (NC) missense based on a probabilistic classifier and weights are assigned to each rare, pathogenic, exomic marker based on the classification scheme wherein the weight for HC>MC>LC>NC. The Sv score for each rare, pathogenic, exomic, missense mutation marker is then augmented according to this weighing scheme.

In step 230, the pathogenic, rare, markers which are deemed to be significant based on the presence or absence of the features in Table 2 are then mapped to the respective genes and the total score of the gene (Sg) is computed based on one or more of the following parameters—the highest Sv score; an arithmetic total of the Sv scores for all markers mapping to the gene; or an arithmetic or geometric mean of the Sv scores of all markers mapping to the gene. Preferably, the Sg score is computed on the basis of the highest Sv score.

Next in step 260, the genes are then tabulated on the basis of the Sg scores, preferably in decreasing order.

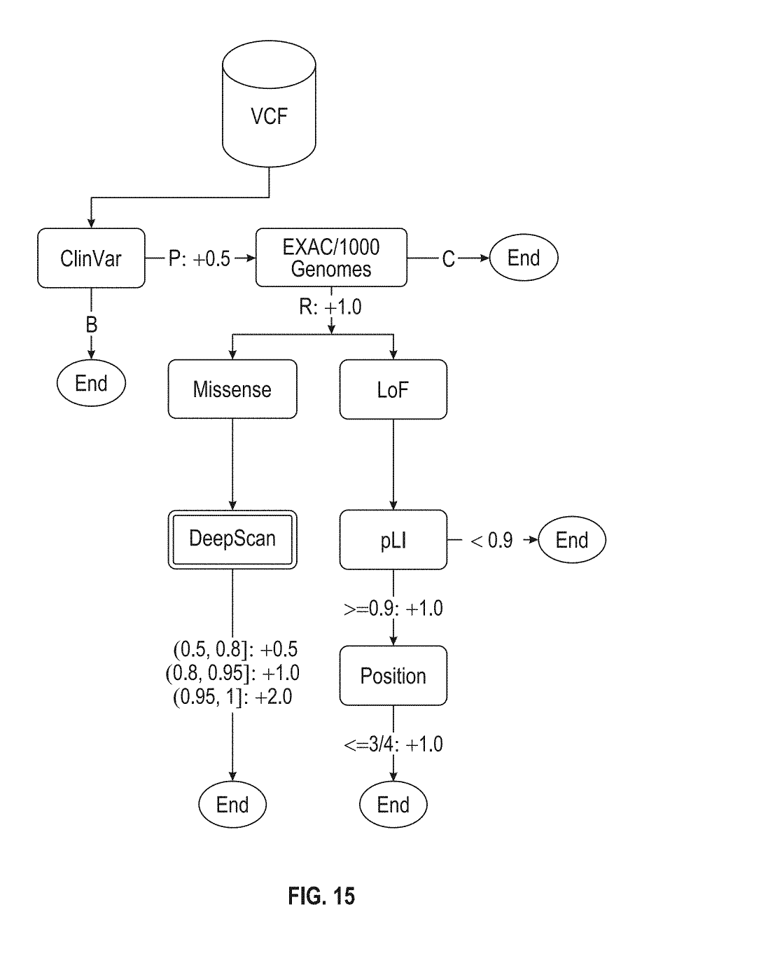

Alternately, the if the selected rare, pathogenic exomic marker comprises an LoF mutation, then a determination is made as to whether the rare, pathogenic marker is LoF tolerant or intolerant based on a probability of LoF intolerant (pLI) score. This is provided in step 235 of FIG. 2. As provided in exemplary FIG. 15, pLI scores are provided by the EXaC database. In some embodiments, QUAL scores provided by the 1000 Genomes database (available on the web at internationalgenome(dot)org) may be used alternately or additionally to the pLI scores. As is known in the art, QUAL represents a Phred-scaled quality score for the assertion made in alt.

Next, in step 240, if the pLI score is below a threshold pLI score, then the variant score (Sv) for that rare, pathogenic, LoF-tolerant exomic marker is unchanged for the rare, pathogenic, LoF-tolerant exomic marker. Typically the pLI score is 0.9, although a larger or smaller threshold pLI score may be used, e.g., 0.95, 0.8 or 0.75 or even 0.7. For rare, pathogenic, LoF-tolerant exomic markers (characterized by pLI that is <threshold pLI), the Sv score is computed in accordance with the foregoing in step 230 and the markers are mapped to the genes and the gene score (Sg) is computed in step 260 in accordance with the foregoing disclosure.

However, if the pLI score is above a threshold pLI score (indicating rare, pathogenic, LoF-intolerant exomic markers), the assigned variant score (Sv) is augmented by a discrete LoF score (>0). Typically, the LoF score is about 1.0 (range of about 0.8 to about 1.2). This is provided in step 245 of FIG. 2.

Next, in step 250, the position of the rare, pathogenic LoF-intolerant exomic marker in the exome sequence is determined. This step may be carried out by referring to the gene sequence, mRNA sequence, or protein sequence. In some embodiments, the exome sequence comprises an unprocessed, e.g., unspliced, mRNA sequence or the cDNA equivalent thereof. In some embodiments, the position of the marker may be determined by counting the total number of units in the macromolecule (e.g., nucleotides in the context of mRNA and/or amino acids in the context of protein) and identifying the position of the marker in reference to the macromolecule. An inquiry is made as to whether the marker is located in the proximal segment of the exome or in the distal segment of the exome. In some embodiments, the proximal segment means the first 80%, the first 70%, the first 60%, the first 50%, the first 40%, the first 30%, or the first 20% of the total units in a linear macromolecule such as mRNA/cDNA or protein. By contrast, the distal segment may mean the last 20%, the last 30%, the last 40%, the last 50%, the last 60% or the last 70% of the total units in a linear macromolecule such as mRNA/cDNA or protein. Herein, the terms “first” and “last” refer to the 5′ end to the 3′ end of the mRNA or cDNA sequence (corresponding to the 3′ end to 5′ end of the gene) or from the N-terminal end to the C-terminal end of the translated protein product (preferably of a processed mature protein).

Next, in step 250, if the rare, pathogenic LoF-intolerant exomic marker is situated in the distal segment of the exome, then the assigned variant score (Sv) for the LoF-intolerant marker is unchanged (no positional score is assigned) and the markers are mapped to the genes and the gene score (Sg) is computed in step 260 in accordance with the foregoing disclosure. However, if the rare, pathogenic LoF-intolerant exomic marker is situated in the proximal segment of the exome, then a positional score is assigned and the Sv score for the rare, pathogenic, LoF-intolerant, proximally-positioned exomic marker is further augmented. Typically, the positional score is about 1.0 (range of about 0.8 to about 1.2). This is provided in step 255 of FIG. 2.

Next, in step 230, a total score for the rare, pathogenic, LoF-intolerant, proximally-positioned exomic marker is calculated, taking into consideration the positional score.

Next in step 260, the markers are mapped to the gene and the Sg score is computed as described in detail above.

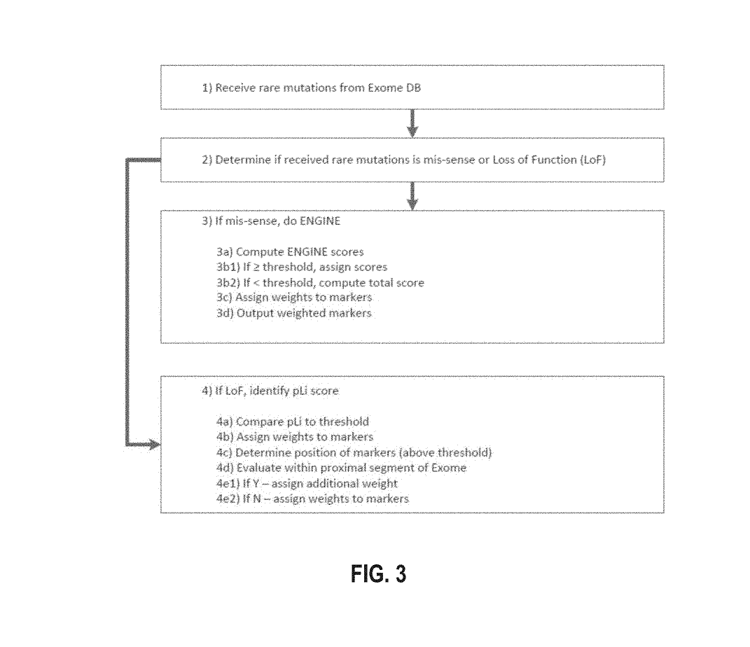

FIG. 3 provides a flowchart of a stand-alone process wherein exomic markers that have already been deemed rare are received from the exome database. Herein, the rare markers are characterized as missense mutations or loss-of-function (LoF) mutations. The missense mutations are analyzed for their significance based on calculations performed by Engine, the details of which are provided in the foregoing paragraphs. The LoF mutations are analyzed on the basis of pLI scores and positional scores, and the significance of the missense mutations are compared to that of the LoF mutations. The comparative assessment is useful since missense mutations are not surveyed with the same rigor as LoF mutations in exomic databases.

The markers for which the aforementioned determinations are carried out may be obtained from subjects. Methods for obtaining exomic data from raw samples, e.g., biological sample such as cells, tissues, biological fluid (e.g., blood, plasma, saliva, semen, pleural fluid), are known in the art. Two common sources of DNA for whole exome sequencing (WES) are whole blood (WB) and immortalized lymphoblastoid cell line (LCL). See, Schafer et al., Genomics, 102(4):270-7, 2013. Other samples may be used. For instance, Poulsen et al. (PLoS One, 11(4):e0153253, 2016) describe exome sequencing of whole-genome amplified neonatal dried blood spot DNA.

Preferably, raw samples for exome sequencing and analysis of exomic markers are obtained from human subjects suffering from a disorder, e.g., a genetic disorder which includes autism spectrum disorder (ASD), epilepsy, seizure, Timothy syndrome, facial dysmorphism, intellectual disability, developmental delay, cancer, or a combination thereof.

### Other Tools

The aforementioned methods are compatible with art-known tools and methods. A detailed overview on the computational tools to analyze and interpret whole exome sequencing data is provided in Hintzsche et al. (Int J Genomics, 2016:7983236, 2016), the disclosure in which is incorporated by reference in its entirety.

In some embodiments, a post frequency-analyzer filter may be optionally applied to further screen the variants identified as rare by the frequency analyzer. For instance, variants that are not expected to be subject to nonsense-mediated decay (NMD) pathway may be removed from the compendium of loss of function variants using the techniques outlined in Kobayashi et al. (Genome Med. 9: 13, 2017). These NMD-negative variants can also be processed through the alternative channel, as mentioned above.

In some embodiments, the variants that are screened using the frequency analyzer and pathogenic analyzer of the disclosure may be further analyzed using sequencing panels, e.g., Illumina's TRUSIGHT inherited disease sequence panel (available on the web at Illumina(dot)com/downloads/trusight_inherited_disease_product_files.html accessed on Jan. 15, 2018). Illumina TRUSIGHT panels may be used to target the exon regions of each gene analyzed. In some embodiments, the variants that are screened using the frequency analyzer and pathogenic analyzer of the disclosure may be further benchmarked using the VARIBENCH metric clusters, which experimentally verifies variants as “pathogenic” and “neutral” (or synonymously benign) datasets. Data may be downloaded and processed from the VARIBENCH website (available on the web at structure(dot)bmc(dot)lu(dot)se/VariBench; accessed on Jan. 15, 2018).

In some embodiments, the variants that are screened using the frequency analyzer and pathogenic analyzer of the disclosure may be further analyzed using Online Mendelian Inheritance in Man (OMIM) catalogue (available on the web at omim(dot)org). A tab-delimited file linking MIM numbers with NCBI Gene IDs, Ensembl Gene IDs, and HGNC Approved Gene Symbols entitled “mim2gene.txt” is available for download via the OMIM website (accessed on Jan. 15, 2018). The file contains allele-specific narrations contain references to other indexed alleles in the same gene that have been detected in compound heterozygous patients.

### IV. Neural Network

By way of illustration only, the disclosure relates to algorithms and software involved in running the diagnostic engine of the disclosure (Engine). In some embodiments, Engine utilizes a classifier that classifies exomic markers on the basis of one or more parameters that give rise to variants that affect function of the encoded protein product. Automated classifiers are an integral part of the fields of data mining and machine learning. There has been widespread use of automated classifying engines to make classifying decisions. Preferably, the classifiers of the disclosure are capable of formalizing genomic data into binary, nominal, rank-ordered, or interval-categorized outcomes. The classifiers of the disclosure can be programmed into computers, robots and artificial intelligence agents for the same types of applications as neural networks, random forests, support vector machines and other such machine learning methods.

Accordingly, in some embodiments, the systems and methods of the disclosure include a support vector machine (SVM) classifier. In some embodiments, the classifier includes logistic regression. In some embodiments, the classifier includes random forest. In some embodiments, the classifier includes naïve Bayes. In some embodiments, the classifier includes gradient boosting. In some embodiments, the classifier includes neural networks.

Preferably the classifier includes neural networks, e.g., convolutional neural network (CNN).

The disclosure further relates to computer-readable storage medium containing a program for detecting tumor markers comprising somatic mutations in a genomic read, the program comprising a layered convolutional neural network (CNN).

As is known in the art, a convolutional neural network (CNN) generally accomplishes an advanced form of processing and classification/detection by first looking for low level features such as, for example, repeat sequences in a read, and then advancing to more abstract (e.g., unique to the type of reads being classified) concepts through a series of convolutional layers. A CNN can do this by passing an image through a series of convolutional, nonlinear, pooling (or downsampling, discussed below), and fully connected layers, and get an output. Again, the output can be a single class or a probability of classes that best describes the image or detects objects on the image.

Regarding layers in a CNN, the first layer is generally a convolutional layer (conv). This first layer will process the read' s representative array using a series of parameters. Rather than processing the image as a whole, a CNN will analyze a collection of image sub-sets using a filter (or neuron or kernel). The sub-sets will include a focal point in the array as well as surrounding points. For example, a filter can examine a series of 5×5 areas (or regions) in a 32×32 image. These regions can be referred to as receptive fields. Since the filter generally will possess the same depth as the input, an image with dimensions of 32×32×3 would have a filter of the same depth (e.g., 5×5×3). The actual step of convolving, using the exemplary dimensions above, would involve sliding the filter along the input image, multiplying filter values with the original pixel values of the image to compute element wise multiplications, and summing these values to arrive at a single number for that examined region of the image.

After completion of this convolving step, e.g., using a 5×5×3 filter, an activation map (or filter map) having dimensions of 28×28×1 will result. For each additional layer used, spatial dimensions are better preserved such that using two filters will result in an activation map of 28×28×2. Each filter will generally have a unique feature it represents that, together, represent the feature identifiers required for the final image output. These filters, when used in combination, allow the CNN to process an image input to detect those features present at each pixel. Therefore, if a filter serves as a curve detector, the convolving of the filter along the image input will produce an array of numbers in the activation map that correspond to high likelihood of a curve (high summed element wise multiplications), low likelihood of a curve (low summed element wise multiplications) or a zero value where the input volume at certain points provided nothing that would activate the curve detector filter. As such, the greater number of filters (also referred to as channels) in the Cony, the more depth (or data) that is provided on the activation map, and therefore more information about the input that will lead to a more accurate output.

Balanced with accuracy of the CNN is the processing time and power needed to produce a result. In other words, the more filters (or channels) used, the more time and processing power needed to execute the Cony. Therefore, the choice and number of filters (or channels) to meet the needs of the CNN method should be specifically chosen to produce as accurate an output as possible while considering the time and power available.

To further enable a CNN to detect more complex features, additional Convs can be added to analyze what outputs from the previous Conv (e.g., activation maps). For example, if a first Conv looks for a basic feature such as a curve or an edge, a second Conv can look for a more complex feature such as shapes, which can be a combination of individual features detected in an earlier Conv layer. By providing a series of Convs, the CNN can detect increasingly higher level features to eventually arrive at a probability of detecting the specific desired object. Moreover, as the Convs stack on top of each other, analyzing the previous activation map output, each Conv in the stack is naturally going to analyze a larger and larger receptive field by virtue of the scaling down that occurs at each Conv level, thereby allowing the CNN to respond to a growing region of pixel space in detecting the object of interest.

A CNN architecture generally consists of a group of processing blocks, including at least one processing block for convoluting an input volume (image) and at least one for deconvolution (or transpose convolution). Additionally, the processing blocks can include at least one pooling block and unpooling block. Pooling blocks can be used to scale down an image in resolution to produce an output available for Conv. This can provide computational efficiency (efficient time and power), which can in turn improve actual performance of the CNN. Those these pooling, or subsampling, blocks keep filters small and computational requirements reasonable, these blocks can coarsen the output (can result in lost spatial information within a receptive field), reducing it from the size of the input by a specific factor.

Unpooling blocks can be used to reconstruct these coarse outputs to produce an output volume with the same dimensions as the input volume. An unpooling block can be considered a reverse operation of a convoluting block to return an activation output to the original input volume dimension. However, the unpooling process generally just simply enlarges the coarse outputs into a sparse activation map. To avoid this result, the deconvolution block densifies this sparse activation map to produce both and enlarged and dense activation map that eventually, after any further necessary processing, a final output volume with size and density much closer to the input volume. As a reverse operation of the convolution block, rather than reducing multiple array points in the receptive field to a single number, the deconvolution block associate a single activation output point with a multiple outputs to enlarge and densify the resulting activation output.

It should be noted that while pooling blocks can be used to scale down an image and unpooling blocks can be used to enlarge these scaled down activation maps, convolution and deconvolution blocks can be structured to both convolve/deconvolve and scale down/enlarge without the need for separate pooling and unpooling blocks.

The pooling and unpooling process can have drawbacks depending on the objects of interest being detected in an image input. Since pooling generally scales down an image by looking at sub-image windows without overlap of windows, there is a clear loss of spatial info as scale down occurs.

A processing block can include other layers that are packaged with a convolutional or deconvolutional layer. These can include, for example, a rectified linear unit layer (ReLU) or exponential linear unit layer (ELU), which are activation functions that examine the output from a Conv in its processing block. The ReLU or ELU layer acts as a gating function to advance only those values corresponding to positive detection of the feature of interest unique to the Conv.

Given a basic architecture, the CNN is then prepared for a training process to hone its accuracy in image classification/detection (of objects of interest). This involves a process called backpropagation (backprop), which uses training data sets, or sample images used to train the CNN so that it updates its parameters in reaching an optimal, or threshold, accuracy. Backpropagation involves a series of repeated steps (training iterations) that, depending on the parameters of the backprop, will either slowly or quickly train the CNN. Backprop steps generally include a forward pass, loss function, backward pass, and parameter (weight) update according to a given learning rate. The forward pass involves passing a training image through the CNN. The loss function is a measure of error in the output. The backward pass determines the contributing factors to the loss function. The weight update involves updating the parameters of the filters to move the CNN towards optimal. The learning rate determines the extent of weight update per iteration to arrive at optimal. If the learning rate is too low, the training may take too long and involve too much processing capacity. If the learning rate is too fast, each weight update may be too large to allow for precise achievement of a given optimum or threshold.

The backprop process can cause complications in training, thus leading to the need for lower learning rates and more specific and carefully determined initial parameters upon start of training. One such complication is that, as weight updates occur at the conclusion of each iteration, the changes to the parameters of the Convs amplify the deeper the network goes. For example, if a CNN has a plurality of Convs that, as discussed above, allows for higher level feature analysis, the parameter update to the first Conv is multiplied at each subsequent Conv. The net effect is that the smallest changes to parameters can have large impact depending on the depth of a given CNN. This phenomenon is referred to as internal covariate shift.

In some embodiments, the CNN of the disclosure comprises a three-layer feed-forward neural network. The CNN models the 473-dimensional encoded features, in which the two hidden layers were both 200-dimensional. The final output neuron indicated whether the input missense variant is pathogenic (1) or benign (0).

The CNN of the disclosure employs a Glorot Normal method to initialize the network weights and ReLU as our activation function. To eliminate the overfitting problem, several techniques were used, including dropout (with rate=0.5), L2 regularization (coefficient=1e-6) and early stopping. Finally, to minimize the binary cross entropy, the Adagrad optimizer (initial learning rate=0.01, ε=1e-6) was performed to train the deep network based on our training samples.

To train Engine, a variety of patients and their matching exomes are first sampled. The goal of the training exercise is to use a training scheme that allows detection of true exomic markers with high sensitivity and also reject candidate markers caused by systemic errors. As described in the Examples, a benchmarked dataset derived from ClinVar and 1000 Genomes project, including 16,930 pathogenic variants from ClinVar and 17,212 benign variants from ClinVar and 1000 Genomes project, was used. The true positive rates, false positive rates and the corresponding receiver operating characteristic (ROC) curve were calculated using MATLAB. It can be seen that the methods of the disclosure performs better than most art-known callers, including, Polyphen. For example, the downside associated with attaining a 90% true positive rate with the methods of the disclosure is about 20% false positive rate; whilst, the false positive rate is about 35% with PolyPhen v2. These data demonstrate that the methods of the disclosure improve precision of calling true positive markers, without the associated drawback of increased noise due to false positives.

In another embodiment, a benchmark dataset from published reports may be used. For example, as described in detail in the Examples, a dataset for autism patients from Lossifov et al. (Nature, 515(7526):216-21, 2014) was used in exemplary methods. Analysis of the dataset was performed using the Engine of the disclosure. Comparisons were made between the outputted results and the output of Mendelian Clinically Applicable Pathogenicity (M-CAP)(Jagadeesh et al., Nature Genetics, 48, 1581-1586, 2016), Combined Annotation Dependent Depletion (CADD)(Kircher et al., Nat Genet. 46(3):310-5, 2014), Polyphen2 (Adzhubei et al., Nat Methods 7(4):248-249, 2010). Engine performed significantly better than most art-known mutation callers in this evaluation.

### V. Profiler

By way of illustration only, and as summary to the following detailed description below, various embodiments herein relate to algorithms and software involved in running an exome analyzer of the disclosure (Profiler). Profiler is capable of performing stringent screening of markers in genetic data of subjects. In general, genetic data comprising exomic markers are obtained using art-known processes, e.g., whole exome sequencing (WES). The genetic data are then evaluated using mutation calling programs e.g., Broad Institute's Genome Analysis Toolkit (GATK), ver. 3.3 (McKenna et al., Genome Res., 20: 1297-1303, 2010). The mutations are functionally annotated using compatible programs such as, e.g., ANNOVAR (Wang et al., Nucleic Acids Res., 38(16): e164, 2010). Subsequently, the mutation called, annotated genetic data are compiled for each patient. In some embodiments, the genetic data comprises a compendium of exomic markers, which are compiled in a variant call format (VCF) file. As is understood in the art, VCF files are used in bioinformatics for storing gene sequence variations. The VCF format has been developed with the advent of large-scale genotyping and DNA sequencing projects, such as the 1000 Genomes Project. Alternately, the compendium may be provided in a general feature format (GFF) containing all of the genetic data. Generally, GFF provides features that are redundant because they are shared across the genomes. In contrast, with VCF, only the variations need to be stored along with a reference genome.

In some embodiments, a subject's sample is sequenced, e.g., using whole exome sequencing (WES), and mutation called and annotated to obtain a sequence file, and the sequence file is processed, e.g., using a tool such as, for example, exome VCF (eVCF) that is available from the NHLBI Grand Opportunity Exome Sequencing Project (ESP).

Then Profiler pipeline is run to score individual coding variants, where a higher score indicates a more pathogenic effect of that variant. The final gene score for each gene in the corresponding gene list, e.g., ACMG and SFARI, is given by the maximum Profiler score across all the variants for that gene.

In some embodiments, the Profiler of the disclosure implements a pipeline scoring system at multiple stages and the pipeline comprises a plurality of blocks and permits that are posited at each stage, wherein if a threshold score for that stage is attained by the marker then the marker is permitted to proceed to the next stage of analysis. A representative pipeline scoring system is presented in FIG. 2 and described in detail in the foregoing paragraphs.

### VI. Clinical Methods

### Diagnosis

In some embodiments, the present disclosure provides a diagnostic test. In one embodiment, the diagnostic test comprises one or more oligonucleotides for use in a hybridization assay. In some embodiments, the diagnostic test comprises one or more devices, tools, and equipment configured to collect a genetic sample from an individual. In some embodiments, tools to collect a genetic sample may include one or more of a swab, a scalpel, a syringe, a scraper, a container, and other devices and reagents designed to facilitate the collection, storage, and transport of a genetic sample. In some embodiments, a diagnostic test may include reagents or solutions for collecting, stabilizing, storing, and processing a genetic sample. Such reagents and solutions for collecting, stabilizing, storing, and processing genetic material are well known by those of skill in the art. In another embodiment, a diagnostic test as disclosed herein, may comprise a microarray apparatus and associated reagents, a flow cell apparatus and associated reagents, a multiplex next generation nucleic acid sequencer and associated reagents, and additional hardware and software necessary to assay a genetic sample for the presence of certain genetic markers and to detect and visualize certain genetic markers.

The disclosure provides diagnosis of at least one or more of the following non-limiting examples of genetic disorders in humans: Timothy's syndrome (based on Sg scores for CACNA1C); Rett's syndrome (MECP2); tuberous sclerosis (based on Sg scores for TSC1 or TSC2 or both); cancer (based on Sg scores for BRCA1, BRCA2, or p53 or a combination thereof); X-linked mental retardation (based on Sg scores for XLMR) syndrome (based on Sg scores for ATRX); autism (based on Sg scores for SHANK3 or PTEN or a combination thereof); Smith-Magenis syndrome (based on association with RAI1); macrocephaly (based on association with PTEN).

### Therapy

In some embodiments, depending on the results of the diagnosis, the subject is selected for treatment for a particular disease. In some embodiments, the subject is selected for the treatment of classic autism. Treatments include, e.g., gene therapy, RNA interference (RNAi), behavioral therapy (e.g., applied behavior analysis (ABA), discrete trial training (DTT), early intensive behavioral intervention (EIBI), pivotal response training (PRT), verbal behavior intervention (VBI), and developmental individual differences relationship-based approach (DIR)), physical therapy, occupational therapy, sensory integration therapy, speech therapy, the picture exchange communication system (PECS), dietary treatment, and drugs (e.g., antipsychotics, antidepressants, anticonvulsants, stimulants).

The disclosure provides therapy of the following genetic disorders: Timothy's syndrome (e.g., gene therapy with CACNA1C); Rett's syndrome (e.g., gene therapy with MECP2); tuberous sclerosis (e.g., gene therapy with TSC1 or TSC2 or both); cancer (e.g., gene therapy with BRCA1, BRCA2, or p53 or a combination thereof); X-linked mental retardation (e.g., gene therapy with XLMR) syndrome (e.g., gene therapy with ATRX); autism (e.g., gene therapy with SHANK3 or PTEN or both); Smith-Magenis syndrome (e.g., gene therapy with RAI1); macrocephaly (e.g., gene therapy with PTEN).

In some embodiments, the subject is selected for the treatment of autism spectrum disorder. Treatments include, e.g., gene therapy, RNAi, occupational therapy, physical therapy, communication and social skills training, cognitive behavioral therapy, speech or language therapy, and drugs (e.g., aripiprazole, guanfacine, selective serotonin reuptake inhibitors (SSRIs), riseridone, olanzapine, naltrexone).

In some embodiments, the subject is selected for the treatment of Rett's disorder. Treatments include, e.g., gene therapy, RNAi, occupational therapy, physical therapy, speech or language therapy, nutritional supplements, and drugs (e.g., SSRIs, anti-psychotics, beta-blockers, anticonvulsants). In some embodiments, the subject is selected for the treatment of CDD. Treatments include, e.g., gene therapy, RNAi, behavioral therapy (e.g., ABA, DTT, EIBI, PRT, VBI, and DIR), sensory enrichment therapy, occupational therapy, physical therapy, speech or language therapy, nutritional supplements, and drugs (e.g., anti-psychotics and anticonvulsants).

In some embodiments, the subject is selected for the treatment of PDD-NOS. Treatments include, e.g., gene therapy, RNAi, behavioral therapy (e.g., ABA, DTT, EIBI, PRT, VBI, and DIR), physical therapy, occupational therapy, sensory integration therapy, speech therapy, PECS, dietary treatment, and drugs (e.g., antipsychotics, anti-depressants, anticonvulsants, stimulants).

In one embodiment, the treatment the subject is selected for is gene therapy to correct, replace, or compensate for a target gene, for example, a wild type allele of one of the genes selected from PTEN.

### EXAMPLES

The structures, materials, compositions, and methods described herein are intended to be representative examples of the disclosure, and it will be understood that the scope of the disclosure is not limited by the scope of the examples. Those skilled in the art will recognize that the disclosure may be practiced with variations on the disclosed structures, materials, compositions and methods, and such variations are regarded as within the ambit of the disclosure.

### Example 1

### Analysis of Benchmarked Dataset Using the Engine of the Disclosure

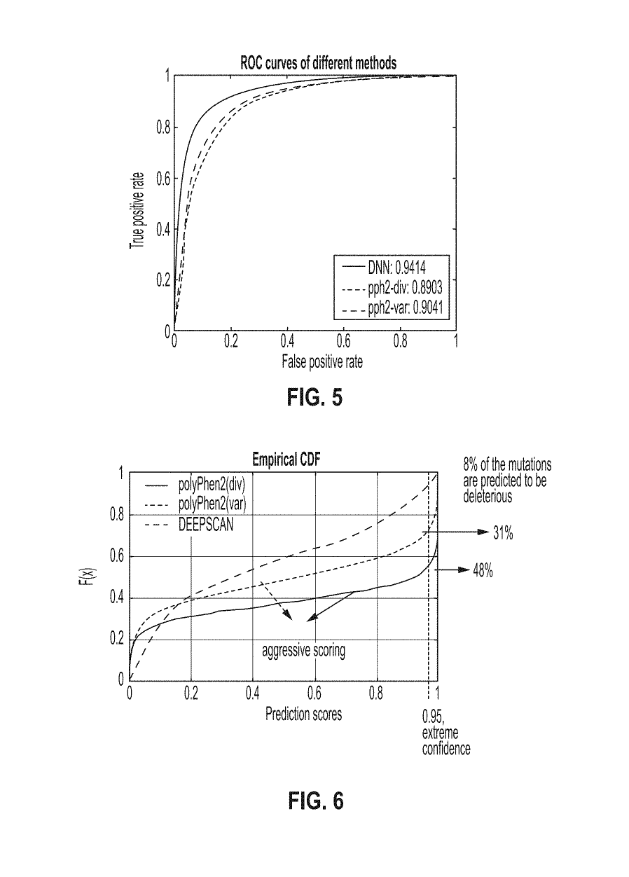

A benchmarked dataset derived from ClinVar and 1000 Genomes project, including 16,930 pathogenic variants from ClinVar and 17,212 benign variants from ClinVar and 1000 Genomes project. Comparative assessment was made using PolyPhen version 2, a software tool that predicts possible impact of an amino acid substitution on the structure and function of a human protein using straightforward physical and comparative considerations Adzhubei et al. (Curr Protoc Hum Genet., Chapter 7:Unit7.20, 2013; and Nat Methods 7(4):248-249, 2010). The true positive rates, false positive rates and the corresponding receiver operating characteristic (ROC) curve were calculated using MATLAB. It can be seen that the Engine of the disclosure performs better than PolyPhen (FIG. 5). For example, the downside associated with attaining a 90% true positive rate with the Engine of the disclosure is about 20% false positive rate; whilst, the false positive rate is about 35% with PolyPhen v2. These data demonstrate that the Engine of the disclosure improve precision of calling true positive markers, without the associated drawback of increased noise due to false positives.

### Example 2

### Analysis of Genetic Markers in Patients with Autism Spectrum Disorder

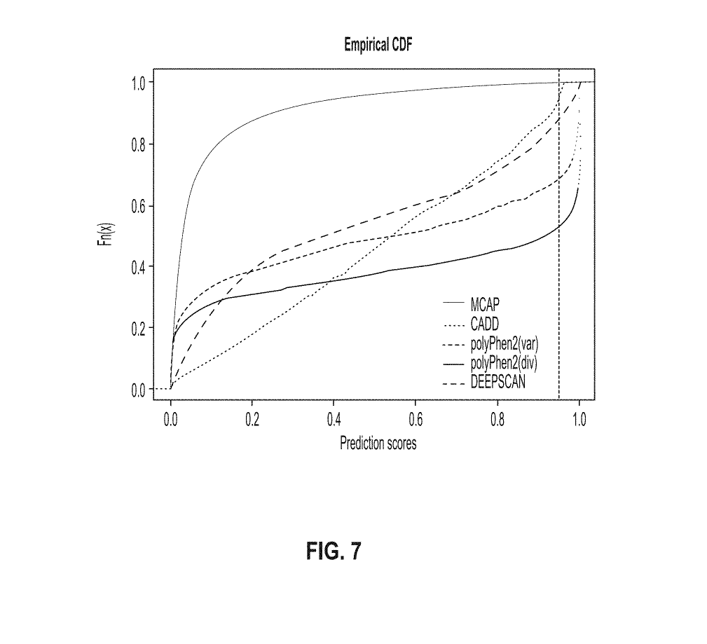

A dataset for patients with autism spectrum disorder was obtained from a published report by Lossifov et al. (Nature, 515(7526):216-21, 2014), the publication and the entire dataset associated therewith being incorporated by reference herein in their entirety. Analysis of the dataset was performed using the Engine of the disclosure. Comparisons were made between the outputted results and the output of Mendelian Clinically Applicable Pathogenicity (M-CAP)(Jagadeesh et al., Nature Genetics, 48, 1581-1586, 2016), Combined Annotation Dependent Depletion (CADD)(Kircher et al., Nat Genet. 46(3):310-5, 2014), Polyphen2 (Adzhubei et al., Nat Methods 7(4):248-249, 2010). Data are shown in FIG. 6 and FIG. 7. It can be seen from the results presented in FIG. 6 that at 95% confidence level, the Engine system of the instant disclosure predicts that about 8% of the mutations in the dataset are deleterious. Polyphen2, in contrast, predicts that nearly half of the mutations in the dataset are deleterious. Even when the comparative assessment was expanded to include M-CAP and CADD, it was found that Engine significantly outperformed these art-known callers. See FIG. 7. Thus, the data in FIG. 6 and FIG. 7 together demonstrate that the Engine of the disclosure permits inclusion of markers (e.g., missense mutations) which would be otherwise excluded from analysis by art-known protein comparative tools.

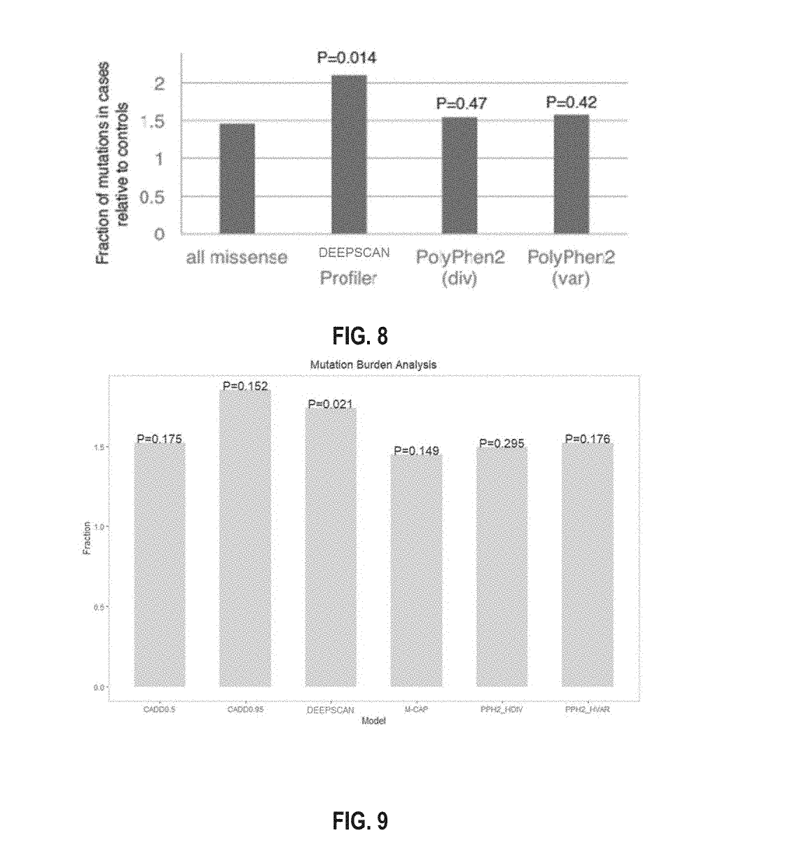

Next, Engine of the disclosure, along with state-of-the art tools were used in analyzing de novo variants (variants appear in subject but not in their parents) in autism spectrum disorder (ASD). The study investigated the de novo missense of cases and controls. As is understood in the art, case probands comprise afflicted subjects (e.g., subjects who currently or at some point in the past had ASD). The controls in contrast comprise siblings who are not afflicted with ASD. It was predicted that the case probands would be associated with a larger number of de novo mutation compared with controls. This ratio between case probands and controls is represented by the “all missense” column in FIG. 5. It should be noted that the analysis did not consider the functional effects (deleterious or benign) of the de novo variants. However, if the de novo missenses are screened using tools like Engine of the disclosure and art-known tools such as PolyPhen, M-CAP or CADD, then sharper comparisons are to be expected. This is because de novo missenses in case probands are expected to be more deleterious than missenses that are present in controls. The data in FIG. 8 show that this hypothesis was experimentally verified using the Engine of the disclosure. Comparative assessments were performed Fisher exact test, wherein a smaller p value indicates a sharper comparison, i.e., a better specificity. It can be seen that the Engine of the disclosure attains a specificity that is not observed with Polyphen. The results are shown in FIG. 8, is significantly superior to Polyphen with regard to analysis of de novo mutations.

The comparative study was expanded to include other analytical tools, e.g., M-CAP and CADD. The data, which are shown in the bar chart of FIG. 9, demonstrate that Engine performs better than all the variation analysis tools, save for CADD (at 95% confidence level).

### Example 3

### Prediction of Single Diseases

Engine was employed in verifying the association between single genes and human diseases and diseases associated with the genes. Benchmarked dataset derived from patients suffering from Timothy's Syndrome, Rett's syndrome; tuberous sclerosis; cancer, and X-lined mental retardation (XLMR), and autism were analyzed.

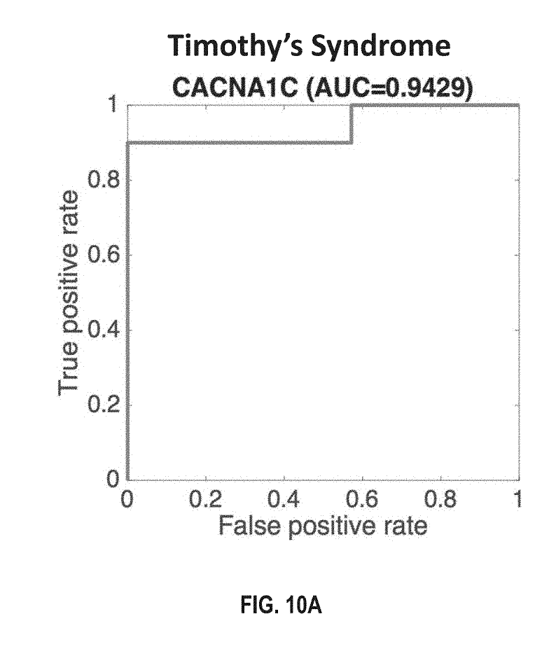

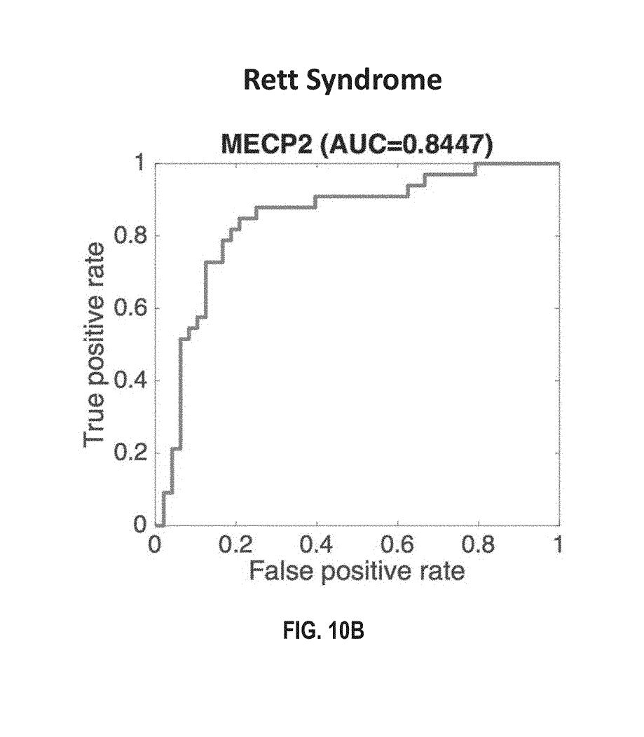

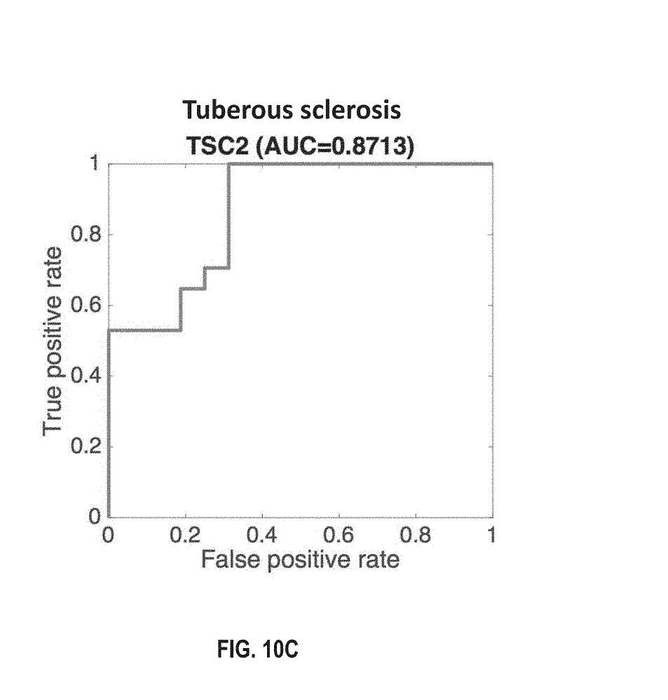

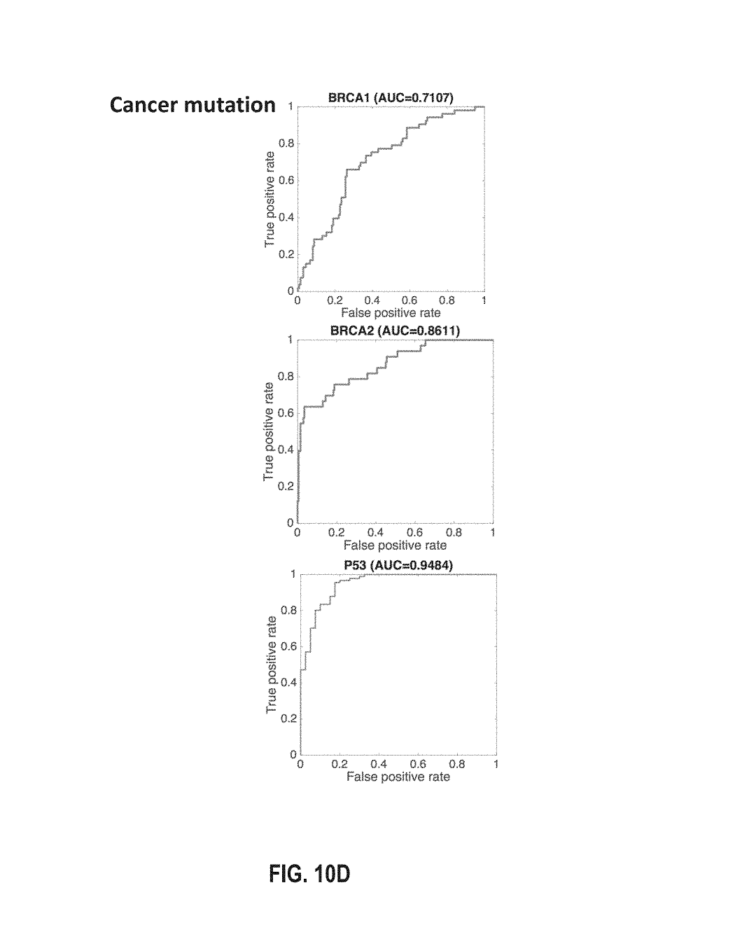

The data were analyzed using a receiver operating characteristic (ROC) curve, which classifies each marker (e.g., missense mutation) in the dataset based on true positive rate and false positive rate. As described previously, the true positive rates, false positive rates and the corresponding receiver operating characteristic (ROC) curve were calculated using MATLAB. Results are shown in FIG. 10, wherein area under the ROC curve indicates the specificity of association between the particular marker and the disease, as analyzed by Engine.

It can be seen that Engine of the disclosure is capable of predicting the association between the various diseases and the mutant gene with a high degree of specificity. In particular, Engine verified that CACNA1C was associated with Timothy's syndrome (FIG. 10A); MECP2 was associated with Rett's syndrome (FIG. 10B); TSC2 was associated with tuberous sclerosis (FIG. 10C); BRCA1, BRCA2 and p53 were all associated with cancer (FIG. 10D). Engine was also able to perfectly predict the association between ATRX and X-lined mental retardation (XLMR), between SKANK1 and autism, and between TSC1 and tuberous sclerosis (AUC=1.0) (ROC curves not shown for perfect association).

### Example 4

### Verification of the Association Between PTEN Mutation and ASD

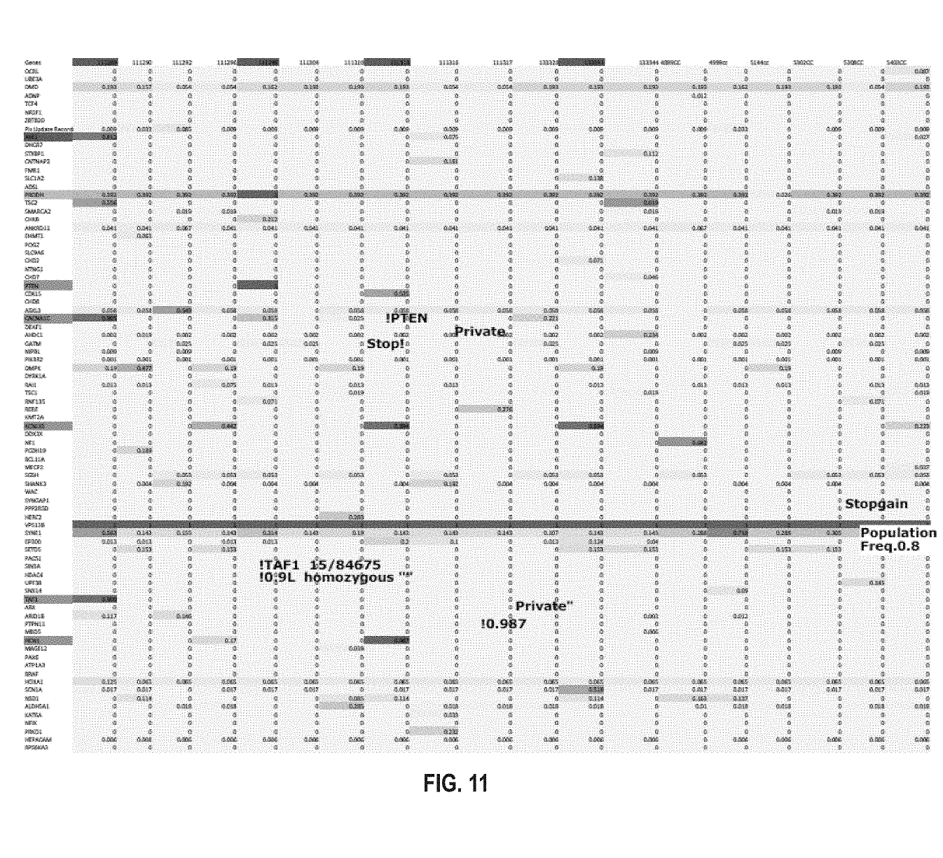

FIG. 11 shows mutational screening result for 19 autism patients, and highlighted one patient with one disrupted gene phosphatase and tensin homolog (PTEN). The PTEN gene disruption comprises a gain a stop codon, resulting in a premature transcript.

### Verification of the Association Between PTEN Mutation and Macrocephaly

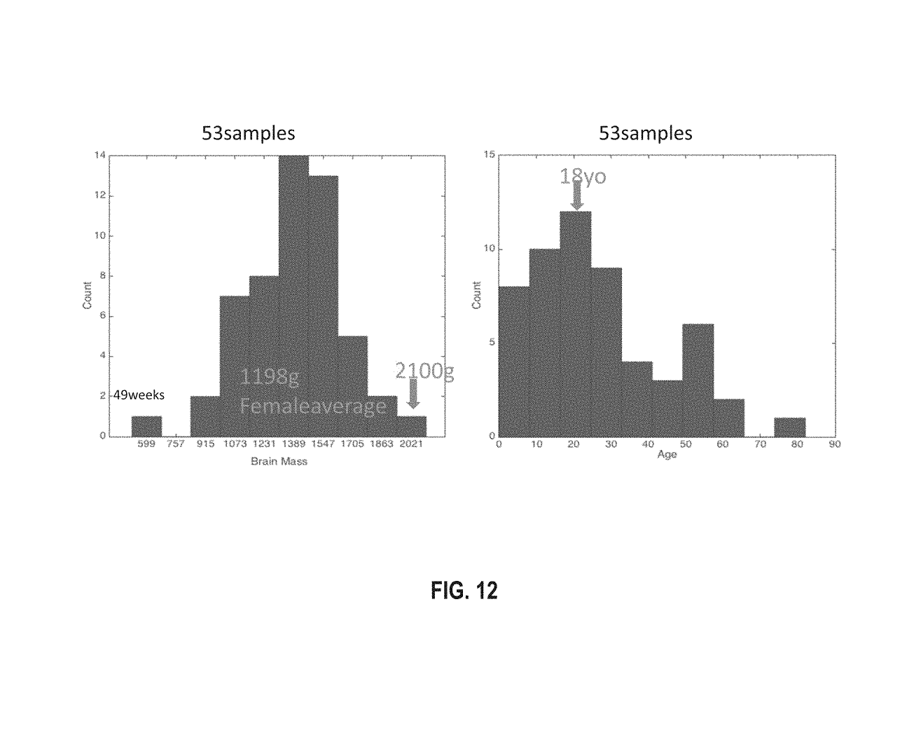

Brain mass was measured in 53 female human subjects and the data on the trait and the age of the subjects was plotted in two histograms (FIG. 12). The chart on the left is a bar-graph of brain mass versus number of subjects falling inside the window; the chart on the right is a bar-graph of the age of the patients versus number of samples falling inside the window. The arrow indicates the brain mass of a subject and the age of the subject. The subject, who was confirmed to have defective PTEN, exhibited substantially increased brain mass, which could not be explained by her age. For example, if age and brain mass were correlated, then the 18-year old female subject would be expected to have an average brain mass and not an enlarged brain mass, as observed.

### Example 5

### Verification of the Association Between RAH and Smith-Magenis Syndrome

Smith Magenis syndrome is caused in most cases (90%) by a 3.7 Mb interstitial deletion in chromosome 17p11.2. The disorder can be caused by a mutation in the RAI1 gene (OMIM: 607672), which is within the Smith Magenis chromosome region. The symptoms of the disease are infantile spasms, cerebral palsy, visual and hearing defects, M-CHAT-R scores of 16 (i.e., >>threshold score of 3). Many cases are undiagnosed.

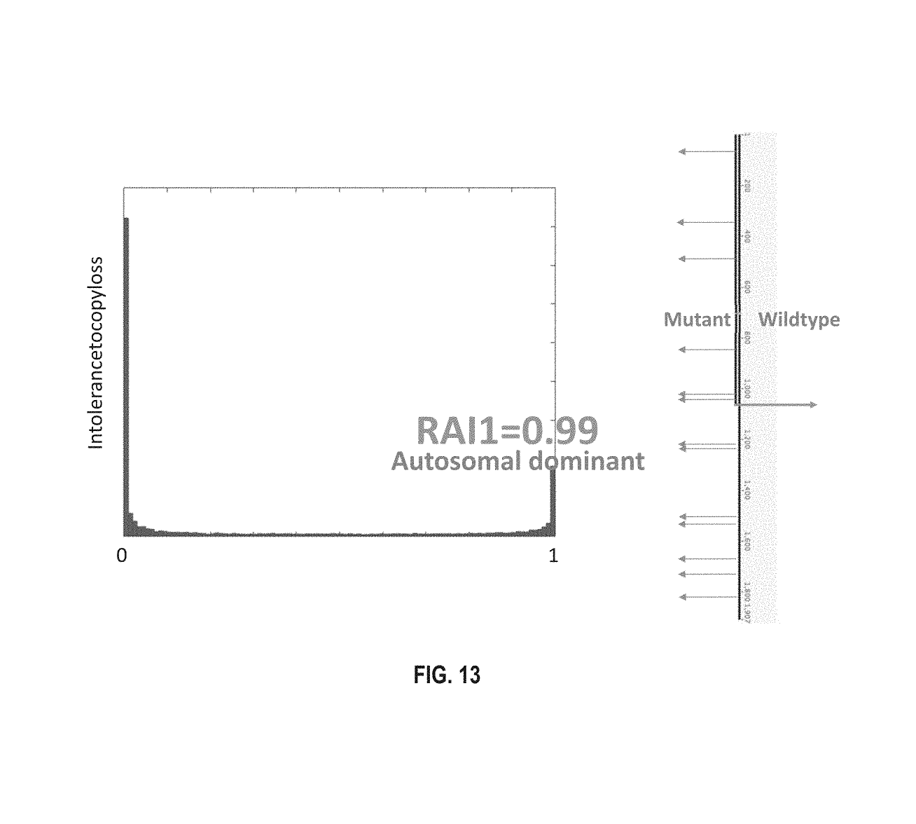

Based on the application of the diagnostic methods and Engine to the patients, a frameshift mutation in the RAI1 gene was identified. The RAI1 mutation is autosomal dominant and is strongly associated with the Smith Magenis phenotypic traits outlined above. The findings are provided in FIG. 13.

### Example 6

### Examination of the Association Between ASD and Genetic Markers

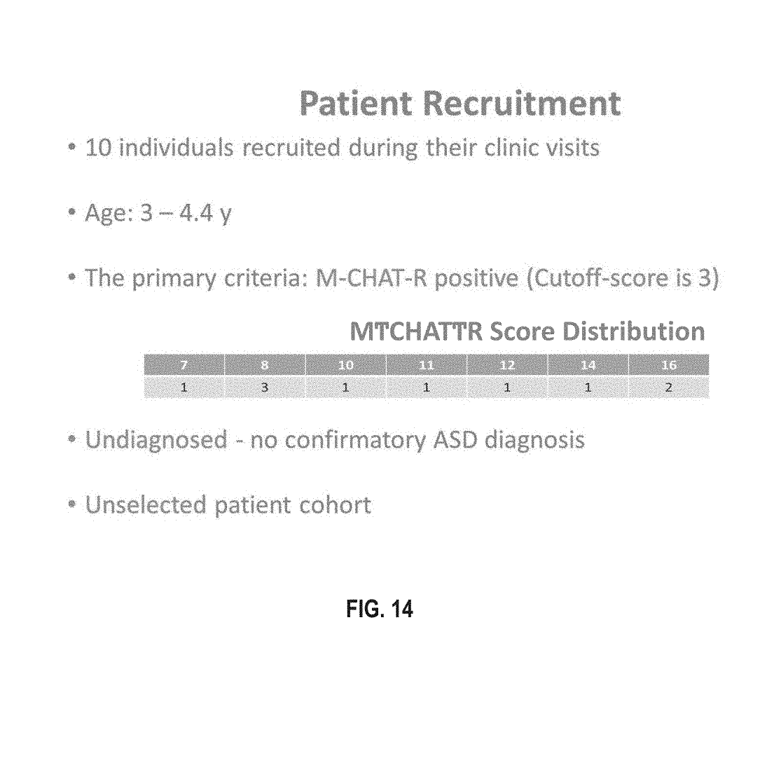

A collaborative project with a children's hospital in China was conducted to benchmark the methods and Engines of the present disclosure. The protocol is summarized in FIG. 14 and follows an institutionally approved ethically compliant procedure for the examination and evaluation of clinical cases. In short, ten individuals with age 3-4.4 years old were recruited for the study. The children recruited for the study had received extremely high M-CHAT-R scores, indicating that they will potentially develop autism in the future. M-CHAT-R is a screening test for autism based on answers provided by a subject to a questionnaire. See NIH Guideline entitled “Revised autism screening tool offers more precise assessment.” (released: Dec. 23, 2013). At the time of the study, the children had not received a clinical diagnosis.

The methods and Engine used in the study can be used to identify genetic markers that allow for early diagnosis of autism in children.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications are referenced. The disclosures of these patents, patent applications, accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

1. A system for diagnosing a genetic disorder, comprising,
(a) a receiving unit for receiving a compendium of markers received from a subject's sample, wherein the markers comprise missense mutations in a read;
(b) a processing unit comprising one or more processors, each of which is configured to execute computer-readable instructions, which when executed, cause the processor to carry out a method or a set of steps comprising
(1) analyzing the compendium of missense mutations for one or more features comprising (I) features relating to protein sequence annotation; (II) features relating to sequence alignment scores; (III) three-dimensional structural features of the encoded protein; (IV) nucleotide sequence context features; or (V) a combination thereof;
(2) assigning a classification score to each missense mutation marker based on the number and/or types of missense features associated therewith;
(3) assigning a variant score (Sv) to each missense mutation based on the classification score;
(4) mapping each missense mutation to one or more genes; and
(5) computing a gene score (Sg) based on the peak, mean or median Sv score of the missense mutation mapped thereto and optionally tabulating the genes in the order of decreasing Sg scores; and
(c) a diagnosing unit which diagnoses the genetic disorder if the optionally tabulated genes with the highest Sg score(s) are associated with the disorder.

2. The system of claim 1, wherein the system comprises
(a) a receiving unit for receiving a compendium of markers received from a subject's sample, wherein the markers further comprise Loss-of-Function (LoF) mutations in a read;
(b) a processing unit comprising one or more processors configured to execute computer-readable instructions, which when executed, cause the processor to carry out a method or a set of steps further comprising
(6) analyzing the compendium of markers comprising LoF mutations for one or more features comprising (VI) probability of intolerant loss of function (pLI) and optionally (VII) proximal positioning of the intolerant LoF mutant marker in the exome sequence;
(7) assigning a variant score (Sv) to each LoF mutant marker based on the pLI score and optionally the proximal position score (PS);
(8) mapping each LoF mutant marker to one or more genes; and
(9) computing a gene score (Sg) based on the peak, mean or median Sv score of the LoF mutant mapped thereto and optionally tabulating the genes in the order of decreasing Sg scores; and
(c) a diagnosing unit which diagnoses the genetic disorder if the optionally tabulated genes with the highest Sg score(s) are associated with the disorder.

3. The system of claim 1, wherein the processor is configured to execute computer-readable instructions, which when executed, cause the processor to carry out a method or a set of steps comprising (1) analyzing the compendium of missense mutations for one or more features comprising
(I) protein sequence annotation feature selected from a categorical feature or an integer feature, wherein the categorical features are selected from (1) UNIPROTKB-database derived substitution SITE annotation; (2) UNIPROTKB-database derived substitution REGION annotation; and (3) Pfam identifier of the query protein; and the integer feature comprises (4) UNIPROTKB or Swiss-PROT-database derived PHAT matrix element for substitutions in the transmembrane region;
(II) sequence alignment score feature which is a real or categorical, wherein the real feature comprises (1) difference of PSIC scores between two amino acid residue variants; (2) PSIC score for wild type amino acid residue; (3) maximum congruency of the mutant amino acid residue to all sequences in multiple alignment; (4) maximum congruency of the mutant amino acid residue to the sequences in multiple alignment with the mutant residue; (5) query sequence identity with the closest homologue deviating from the wild type amino acid residue; or an integer feature which is (6) number of residues at the substitution position in multiple alignment;
(III) three-dimensional structural features of the encoded protein, which are real features, categorical features, or integer features, wherein the real features are selected from (1) sequence identity between query sequence and aligned PDB sequence; (2) normalized accessible surface area; (3) change in solvent accessible surface propensity; (4) normalized B-factor (temperature factor) for the residue; (5) closest residue contact with a heteroatom, Å; (6) closest residue contact with other chain; Å; and (7) closest residue contact with a critical site, Å; and wherein the category features are selected from (8) DSSP secondary structure assignment; and (9) region of the Ramachandran map derived from the residue dihedral angles; and wherein the integer feature selected from (10) change in residue side chain volume; (11) number of hydrogen sidechain-sidechain and sidechain-mainchain bonds formed by the residue; (12) number of residues in contacts with heteroatoms, average per homologous PDB chain; (13) number of residue contacts with other chains, average per homologous PDB chain; and (14) number of residue contacts with critical sites, average per homologous PDB chain; and/or
(IV) nucleotide sequence context features, which are binary features, categorical features, or integer features, wherein, the binary features comprise (1) assessment of transversions;
wherein categorical features comprise (2) assessment of position of the substitution within a codon; or (3) substitution changes CpG context; and wherein the integer feature comprises (4) assessment of the substitution distance from closest exon/intron junction.

4. The system of claim 3, wherein the diagnosing unit comprises a neural network which is capable of identifying markers associated with the disorder from a training dataset generated from a genetic data of a patient diagnosed with the disorder or a subject related thereto, wherein the training dataset comprises a compendium of markers that are prognostic of the disease.

5. A method for diagnosing a genetic disorder, comprising,
(a) receiving in a compendium of markers received from a subject's sample, wherein the markers comprise missense mutations in an read;
(b) implementing a plurality of computer-assisted analytical steps comprising
(1) analyzing the compendium of missense mutations for one or more features comprising (I) features relating to protein sequence annotation; (II) features relating to sequence alignment scores; (III) three-dimensional structural features of the encoded protein; (IV) nucleotide sequence context features; or (V) a combination thereof;
(2) assigning a classification score to each missense mutation marker based on the number and/or types of missense features associated therewith;
(3) assigning a variant score (Sv) to each missense mutation based on the classification score;
(4) mapping each missense mutation to one or more genes; and
(5) computing a gene score (Sg) based on the peak, mean or median Sv score of the missense mutation mapped thereto and optionally tabulating the genes in the order of decreasing Sg scores; and
(c) diagnosing the genetic disorder if the optionally tabulated genes with the highest Sg score(s) are associated with the disorder.

6. The method of claim 5, wherein step (a) further comprises receiving compendium of markers received from a subject's sample, wherein the markers further comprise Loss-of-Function (LoF) mutations in an read; step (b) further implementing a plurality of computer-assisted analytical steps comprising (6) analyzing the compendium of markers comprising LoF mutations for one or more features comprising (VI) probability of intolerant loss of function (pLI) and optionally (VII) proximal positioning of the intolerant LoF mutant marker in the exome sequence; (7) assigning a variant score (Sv) to each LoF mutant marker based on the pLI score and optionally the proximal position score (PS); (8) mapping each LoF mutant marker to one or more genes; and (9) computing a gene score (Sg) based on the peak, mean or median Sv score of the LoF mutant mapped thereto and optionally tabulating the genes in the order of decreasing Sg scores; and step (c) further comprises diagnosing the genetic disorder if the optionally tabulated genes with the highest Sg score(s) are associated with the disorder.

7. The method of claim 5, wherein the computer-assisted method comprises analyzing the compendium of missense mutations for one or more features comprising
(I) protein sequence annotation feature selected from a categorical feature or an integer feature, wherein the categorical features are selected from (1) UNIPROTKB-database derived substitution SITE annotation; (2) UNIPROTKB-database derived substitution REGION annotation; and (3) Pfam identifier of the query protein; and the integer feature comprises (4) UNIPROTKB or Swiss-PROT-database derived PHAT matrix element for substitutions in the transmembrane region;
(II) sequence alignment score feature which is a real or categorical, wherein the real feature comprises (1) difference of PSIC scores between two amino acid residue variants; (2) PSIC score for wild type amino acid residue; (3) maximum congruency of the mutant amino acid residue to all sequences in multiple alignment; (4) maximum congruency of the mutant amino acid residue to the sequences in multiple alignment with the mutant residue; (5) query sequence identity with the closest homologue deviating from the wild type amino acid residue; or an integer feature which is (6) number of residues at the substitution position in multiple alignment;
(III) three-dimensional structural features of the encoded protein, which are real features, categorical features, or integer features, wherein the real features are selected from (1) sequence identity between query sequence and aligned PDB sequence; (2) normalized accessible surface area; (3) change in solvent accessible surface propensity; (4) normalized B-factor (temperature factor) for the residue; (5) closest residue contact with a heteroatom, Å; (6) closest residue contact with other chain; Å; and (7) closest residue contact with a critical site, Å; and wherein the category features are selected from (8) DSSP secondary structure assignment; and (9) region of the Ramachandran map derived from the residue dihedral angles; and wherein the integer feature selected from (10) change in residue side chain volume; (11) number of hydrogen sidechain-sidechain and sidechain-mainchain bonds formed by the residue; (12) number of residues in contacts with heteroatoms, average per homologous PDB chain; (13) number of residue contacts with other chains, average per homologous PDB chain; and (14) number of residue contacts with critical sites, average per homologous PDB chain; and/or
(IV) nucleotide sequence context features, which are binary features, categorical features, or integer features, wherein, the binary features comprise (1) assessment of transversions;
wherein categorical features comprise (2) assessment of position of the substitution within a codon; or (3) substitution changes CpG context; and wherein the integer feature comprises (4) assessment of the substitution distance from closest exon/intron junction.

8. The method of claim 7, wherein the diagnosing step (c) comprises implementing a neural network to analyze the markers, wherein the neural network is trained with a dataset generated from a genetic data of a patient diagnosed with the disorder or a subject related thereto.

9. A method for determining markers linked to a disorder in a subject, comprising
(A) receiving a dataset comprising one or more variant markers, wherein the dataset is obtained by sequencing a biological sample comprising nucleic acid molecules from a subject afflicted with the disorder;
(B) analyzing each variant marker on the basis of a plurality of scores dispensed by a pipeline scoring system, the scoring system comprising:
(1) assessing a pathogenic significance of each variant marker based on a clinical significance score thereof in a first database of clinically significant nucleic acid variations, wherein variant marker assessed to be clinically significant are assigned a clinical significant score (Sv) and are selected for further analysis in the pipeline;
(2) assessing a frequency of each clinically-significant variant marker of (1) on the basis of frequency score thereof in a second database of nucleic acid variations, wherein clinically-significant variant markers assessed to be rare are assigned an augmented Sv score and are selected for further analysis in the pipeline;
(3) binning each rare, clinically-significant variant marker of (2) on the basis of a severity of the variation, wherein variant markers having rare, clinically-significant, loss-of-function (LoF) variations are binned separately from variant markers having rare, clinically-significant, missense variations; wherein the pipeline of a first bin comprises
(4)(a) assessing each LoF variant markers of the first on the basis of probability of loss-of-function intolerant (pLI) score thereof in a third database, wherein rare, clinically-significant, LoF variant markers having pLI scores above a threshold are assigned a further augmented Sv score and are selected for further analysis in the pipeline;
(4)(b) assessing each selected LoF variant markers of 4(a) on the basis of position of variation, wherein selected LoF variant markers having variations located in the proximal end of the coding nucleic acid sequences are assigned a still further augmented Sv score;
and the pipeline of the second bin comprises
(4)(c) assessing each missense variant markers of the second bin via a neural network which weighs each missense variant marker and further augments the Sv score thereof based on the weight, wherein the weighing step comprises analyzing each missense variant on the basis of at least one feature selected from (I) protein sequence annotation; (II) sequence alignment scores; (III) 3-dimensional structural features of the encoded protein; (IV) nucleotide sequence context features; or (V) a combination thereof;
(C) mapping each variant coding nucleic acid to a gene and computing a gene score (S_{g}) based on the Sv value of one or more variants mapped thereto; and
(D) selecting genes whose S_{g} scores are above a threshold level as being linked to the disorder.

10. The method of claim 9, wherein the weighing step comprises analyzing each missense variant on the basis of (I) protein sequence annotation feature selected from a categorical feature or an integer feature, wherein the categorical features are selected from (1) UNIPROTKB-database derived substitution SITE annotation; (2) UNIPROTKB-database derived substitution REGION annotation; (3) Pfam identifier of the query protein; and the integer feature comprises (4) UNIPROTKB or Swiss-PROT-database derived PHAT matrix element for substitutions in the transmembrane region.

11. The method of claim 9, wherein the weighing step comprises analyzing each missense variant on the basis of (II) sequence alignment score feature which is a real or categorical, wherein the real feature comprises (1) difference of PSIC scores between two amino acid residue variants; (2) PSIC score for wild type amino acid residue; (3) maximum congruency of the mutant amino acid residue to all sequences in multiple alignment; (4) maximum congruency of the mutant amino acid residue to the sequences in multiple alignment with the mutant residue; (5) query sequence identity with the closest homologue deviating from the wild type amino acid residue; or an integer feature which is (6) number of residues at the substitution position in multiple alignment.

12. The method of claim 9, wherein the weighing step comprises analyzing each missense variant on the basis of (III) three-dimensional structural features of the encoded protein, which are real features, categorical features, or integer features, wherein the real features are selected from (1) sequence identity between query sequence and aligned PDB sequence; (2) normalized accessible surface area; (3) change in solvent accessible surface propensity; (4) normalized B-factor (temperature factor) for the residue; (5) closest residue contact with a heteroatom, Å; (6) closest residue contact with other chain; Å; and (7) closest residue contact with a critical site, Å; wherein the category features are selected from (8) DSSP secondary structure assignment; and (9) region of the Ramachandran map derived from the residue dihedral angles; and wherein the integer feature selected from (10) change in residue side chain volume; (11) number of hydrogen sidechain-sidechain and sidechain-mainchain bonds formed by the residue; (12) number of residues in contacts with heteroatoms, average per homologous PDB chain; (13) number of residue contacts with other chains, average per homologous PDB chain; and (14) number of residue contacts with critical sites, average per homologous PDB chain.

13. The method of claim 9, wherein the weighing step comprises analyzing each missense variant on the basis of (IV) nucleotide sequence context features, which are binary features, categorical features, or integer features,
wherein, the binary features comprise (1) assessment of transversions;
wherein categorical features comprise (2) assessment of position of the substitution within a codon; or (3) substitution changes CpG context; and
wherein the integer feature comprises (4) assessment of the substitution distance from closest exon/intron junction.

14. The method of claim 9, wherein the subject is a human subject and the disorder comprises autism spectrum disorder (ASD), epilepsy, seizure, Timothy syndrome, facial dysmorphism, intellectual disability, developmental delay, cancer, or a combination thereof.

15. The method of claim 9, wherein the variant exomic sequence comprises a DNA or an RNA sequence which encodes a polypeptide.

16. The method of claim 9, wherein the receiving step comprises whole exome sequencing of the subject's exome, optional mutation calling and further optionally annotating variants.

17. The method of claim 16, wherein the mutation calling step comprises employing genomic analysis toolkit software (GATK) and the annotating step comprises employing Annotate Variation software (ANNOVAR).

18. The method of claim 9, wherein the biological sample comprises a cell sample containing genomic DNA or total mRNA encoding the subject's proteome.

19. The method of claim 9, wherein the pipeline scoring system is implemented at multiple stages and the pipeline comprises a plurality of blocks and permits that are posited at each stage, wherein if a threshold score for that stage is attained by the marker then the marker is permitted to proceed to the next stage of analysis.

20. The method of claim 9, wherein the clinical significance of the marker is assessed based on the score assigned to the marker by NCBI CLINVAR database.

21-99. (canceled).
